(19) United States
(12) Patent Application Publication
(10) Pub. No.: US 2004/0211010 A1
(43) Pub. Date: Oct. 28, 2004

(54) Dye composition comprising a cationic tertiary para-phenylenediamine and a polyol ester, processes therefor and uses thereof

(75) Inventors: Jean Cotteret, Verneuil Sur Seine (FR); Alain Lagrange, Coupvray (FR)

Correspondence Address: Mark B. Wilson, Fulbright & Jaworski L.L.P., Suite 2400, 600 Congress Avenue, Austin, TX, 78701, US

(73) Assignee: L'OREAL, Paris (FR)

(21) Appl. No.: 10/734,612

(22) Filed: Dec. 12, 2003

Related U.S. Application Data
(60) Provisional application No. 60/444,625, filed on Feb. 4, 2003.

(30) Foreign Application Priority Data
Dec. 13, 2002 (FR) 02/15776

(51) Int. Cl. A61K 7/13

(52) U.S. Cl. 8/405

(57) ABSTRACT

The present patent application relates to a dye composition for dyeing keratin fibers, in particular human keratin fibers such as the hair, comprising, in a medium that is suitable for dyeing, at least one cationic tertiary para-phenylenediamine containing a pyrrolidine nucleus, and at least one polyol ester.

The invention also relates to the dyeing process using this composition.

### CROSS REFERENCE TO RELATED APPLICATIONS

This application claims priority to French Application No. 02/15776 filed 13 Dec. 2003, and further claims the benefit of U.S. Provisional Application No. 60/444,625 filed 04 Feb. 2003, the entire disclosures of which are incorporated herein by reference in their entirety.

### BACKGROUND OF THE INVENTION

The present patent application relates to a dye composition for dyeing keratin fibres, in particular human keratin fibres such as the hair, comprising, in a medium that is suitable for dyeing, at least one cationic tertiary para-phenylenediamine containing a pyrrolidine nucleus, and at least one polyol ester.

The invention also relates to the use of this composition for dyeing keratin fibres, and to the dyeing process using this composition.

It is known practice to dye keratin fibres, and in particular human hair, with dye compositions containing oxidation dye precursors, which are generally known as oxidation bases, such as ortho- or para-phenylenediamines, ortho- or para-aminophenols, and heterocyclic compounds. These oxidation bases are colourless or weakly coloured compounds that, when combined with oxidizing products, may give rise to coloured compounds by a process of oxidative condensation.

It is also known that the shades obtained with these oxidation bases may be varied by combining them with couplers or coloration modifiers, the latter being chosen especially from aromatic meta-diamines, meta-aminophenols, meta-diphenols and certain heterocyclic compounds such as indole compounds.

The variety of molecules used as oxidation bases and couplers allows a wide range of colours to be obtained.

The “permanent” coloration obtained using these oxidation dyes must moreover satisfy a certain number of requirements. Thus, it must have no toxicological drawback, it must be able to produce shades in the desired intensity, and it must show good resistance to external agents such as light, bad weather, washing, permanent-waving, perspiration and rubbing.

The dyes must also be able to cover grey hair and, finally, they must be as unselective as possible, i.e. they must produce the smallest possible differences in coloration along the same keratin fibre, which is generally differently sensitized (i.e. damaged) between its end and its root.

It has already been proposed, in patent application WO 02/45675, to use compositions for the oxidation dyeing of keratin fibres, comprising a cationic tertiary para-phenylenediamine containing a pyrrolidine nucleus.

These cationic tertiary para-phenylenediamines containing a pyrrolidine nucleus lead to compositions whose harmlessness is generally considered as being better than that of compositions containing standard para-phenylenediamines. However, the shades obtained when these compositions are used are markedly weaker and markedly more selective, i.e. the shades obtained show large variations in coloration depending on the degree of sensitization of various hairs or of the various regions of the same hair. The fastness of these shades may also vary greatly depending on the degree of sensitization. In addition, the colorations obtained are also often more grey, i.e. less chromatic.

### SUMMARY OF THE INVENTION

The Applicant has just discovered, surprisingly and advantageously, that it is possible to obtain novel compositions for the dyeing of keratin fibres, in particular human keratin fibres such as the hair, which are capable of overcoming the abovementioned drawbacks and of producing varied, chromatic, strong, attractive, relatively unselective shades that show good resistance to the various attacking factors to which the fibres may be subjected, by combining in a composition at least one cationic tertiary para-phenylenediamine containing a pyrrolidine nucleus and at least one polyol ester. In addition, these compositions have a good toxicological profile.

A subject of the invention is also a dyeing process using this composition, and also a multi-compartment device or dyeing “kit”.

Another subject of the invention is the use of the composition of the present invention for dyeing keratin fibres, in particular human keratin fibres such as the hair.

The composition of the present invention makes it possible in particular to obtain a chromatic, very strong, relatively unselective and fast coloration of keratin fibres, while at the same time avoiding the degradation of these fibres.

For the purposes of the present invention, the expression “cationic tertiary para-phenylenediamine containing a pyrrolidine nucleus” means a para-phenylenediamine containing an NH₂ group and, para to this, a disubstituted amine function whose substitutions form a pyrrolidine nucleus with the nitrogen atom, the molecule containing at least one quaternized nitrogen atom.

In the context of the present invention, the term “alkyl” means linear or branched radicals, for example methyl, ethyl, n-propyl, isopropyl, butyl, etc. An alkoxy radical is a radical alk-O, the alkyl radical having the definition given above. The term “halogen” preferably denotes Cl, Br, I or F.

Among the cationic tertiary para-phenylenediamines containing a pyrrolidine nucleus that may be used in the composition according to the present invention, mention may be made especially of the compounds of formula (I) below, and the addition salts thereof:

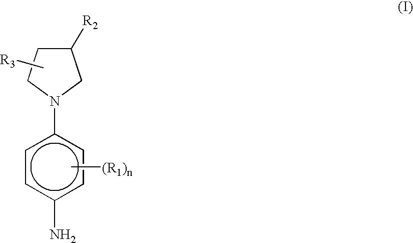

in which

tranges from 0 to 4, it being understood that when n is greater than or equal to 2, then the radicals R₁ may be identical or different,

R₁ represents a halogen atom; a C₁-C₆ aliphatic or alicyclic, saturated or unsaturated hydrocarbon-based chain, the chain possibly being interrupted with one or more oxygen, nitrogen, silicon or sulphur atoms or with an SO₂ group, and possibly being substituted with one or more hydroxyl or amino radicals; an onium radical Z, the radical R₁ not comprising a peroxide bond or diazo, nitro or nitroso radicals,

R₂ represents an onium radical Z or a radical —X—C═NR₈—NR₉R₁₀ in which X represents an oxygen atom or a radical —NR₁₁ and R₈, R₉, R₁₀ and R₁₁ represent a hydrogen atom, a C₁-C₄ alkyl radical or a C₁-C₄ hydroxyalkyl radical,

R₃ represents a hydrogen atom or a hydroxyl radical.

The term “onium” denotes a quaternary radical of a nitrogenous base.

By way of example, R₁ may be a chlorine atom or a methyl, ethyl, isopropyl, vinyl, allyl, methoxymethyl, hydroxyethyl, 1-carboxymethyl, 1-aminomethyl, 2-carboxyethyl, 2-hydroxyethyl, 3-hydroxypropyl, 1,2-dihydroxyethyl, 1-hydroxy-2-aminoethyl, 1-amino-2-hydroxyethyl, 1,2-diaminoethyl, methoxy, ethoxy, allyloxy or 2-hydroxyethyloxy radical.

In particular, n is equal to 0.

In formula (I), when n is equal to 1, R₁ is preferably a halogen atom; a C₁-C₆ aliphatic or alicyclic, saturated or unsaturated hydrocarbon-based chain, one or more carbon atoms possibly being replaced with an oxygen, nitrogen, silicon or sulphur atom or with an SO₂ group, the radical R₁ not comprising a peroxide bond or diazo, nitro or nitroso radicals. Preferably, R₁ is chosen from chlorine, bromine and a C₁-C₄ alkyl, C₁-C₄ hydroxyalkyl, C₁-C₄ aminoalkyl, C₁-C₄ alkoxy or C₁-C₄ hydroxyalkoxy radical. By way of example, R₁ is chosen from a methyl, hydroxymethyl, 2-hydroxyethyl, 1,2-dihydroxyethyl, methoxy, isopropyloxy or 2-hydroxyethoxy radical.

The radical R₂ of formula (I) is, according to one particular embodiment, the onium radical Z corresponding to formula (II)

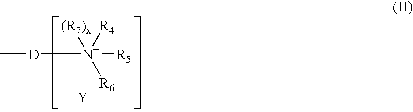

in which

D is a single bond or a linear or branched C₁-C₁₄ alkylene chain which may be interrupted with one or more hetero atoms chosen from oxygen, sulphur and nitrogen, and which may be substituted with one or more hydroxyl, C₁-C₆ alkoxy or amino radicals, and which may bear one or more ketone functions;

R₄, R₅ and R₆, taken separately, represent a C₁-C₁₅ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical; a benzyl radical; a C₁-C₆ amidoalkyl radical; a tri(C₁-C₆)alkylsilane(C₁-C₆)alkyl radical; a C₁-C₆ aminoalkyl radical; a C₁-C₆ aminoalkyl radical in which the amine is mono- or disubstituted with a C₁-C₄ alkyl, (C₁-C₆)alkylcarbonyl, amido or (C₁-C₆)alkylsulphonyl radical; or

R₄, R₅ and R₆ together, in pairs, form, with the nitrogen atom to which they are attached, a saturated 4-, 5-, 6- or 7-membered carbon-based ring optionally containing one or more hetero atoms such as, for example, an azetidine ring, a pyrrolidine ring, a piperidine ring, a piperazine ring or a morpholine ring, the cationic ring possibly being substituted with a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a (C₁-C₆)alkylcarbonyl radical, a thio (—SH) radical, a C₁-C₆ thioalkyl (—R—SH) radical, a (C₁-C₆)alkylthio radical, an amino radical, an amino radical mono- or disubstituted with a (C₁-C₆)alkyl, (C₁-C₆)alkylcarbonyl, amido or (C₁-C₆)alkylsulphonyl radical;

R₇ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ tri-fluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkyl-sulphonamido(C₁-C₆)alkyl radical;

x is 0 or 1,

when x=0, then the linker arm is attached to the nitrogen atom bearing the radicals R₄ to R₆,

when x=1, then two of the radicals R₄ to R₆ form, together with the nitrogen atom to which they are attached, a 4-, 5-, 6- or 7-membered saturated ring and D is linked to a carbon atom of the saturated ring;

Y is a counterion.

In formula (II), when x is equal to 0, then R₄, R₅ and R₆, separately, are preferably chosen from a C₁-C₆ alkyl radical, a C₁-C₄ monohydroxyalkyl radical, a C₂-C₄ polyhydroxyalkyl radical, a (C₁-C₆)alkoxy(C₁-C₄)alkyl radical, a C₁-C₆ amidoalkyl radical, a tri(C₁-C₆)-alkylsilane(C₁-C₆)alkyl radical, or R₄ and R₅ together form an azetidine, pyrrolidine, piperidine, piperazine or morpholine ring, R₆ being chosen in this case from a C₁-C₆ alkyl radical; a C₁-C₆ monohydroxyalkyl radical; a C₂-C₆ polyhydroxyalkyl radical; a C₁-C₆ aminoalkyl radical, an aminoalkyl radical mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

When x is equal to 1, then R₇ is preferably chosen from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N-(C₁-C₆)alkylcarbamyl(C₁-C₆)alkyl radical; R₄ and R₅ together form an azetidine, pyrrolidine, piperidine, piperazine or morpholine ring, R₆ being chosen in this case from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

In formula (II), D is preferably a single bond or an alkylene chain that may be substituted.

When the radical R₂ corresponds to formula (II), it is preferably a trialkylammonium radical, the alkyl radicals of which may be substituted.

According to a second embodiment, the radical R₂ represents the onium radical Z corresponding to formula (III)

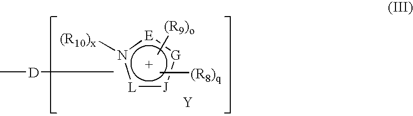

in which

D is a single bond or a linear or branched C₁-C₁₄ alkylene chain that may be interrupted with one or more hetero atoms chosen from oxygen, sulphur and nitrogen, and that may be substituted with one or more hydroxyl, C₁-C₆ alkoxy or amino radicals, and that may bear one or more ketone functions;

the ring members E, G, J and L, which may be identical or different, represent a carbon, oxygen, sulphur or nitrogen atom to form a pyrrole, pyrazole, imidazole, triazole, oxazole, isoxazole, thiazole or isothiazole ring,

q is an integer between 0 and 4 inclusive;

o is an integer between 0 and 3 inclusive;

q+o is an integer between 0 and 4;

the radicals R₈, which may be identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical mono- or disubstituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₈ are borne by a carbon atom,

the radicals R₉, which may be identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical or a benzyl radical; it being understood that the radicals R₉ are borne by a nitrogen atom,

R₁₀ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical, a C₁-C₆ aminoalkyl radical in which the amine is substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;

x is 0 or 1

when x=0, the linker arm D is attached to the nitrogen atom,

when x=1, the linker arm D is attached to one of the ring members E, G, J or L,

Y is a counterion.

The ring members E, G, J and L preferably form an imidazole ring.

Among the radicals R₂ of formula (III), the ones that are preferred are the radicals in which x is equal to 0 and D is a single bond or an alkylene chain that may be substituted.

According to a third embodiment, R₂ represents the onium radical Z corresponding to formula (IV)

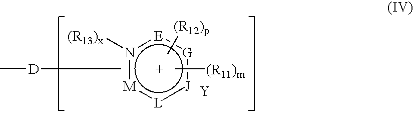

in which:

D is a single bond or a linear or branched C₁-C₁₄ alkylene chain which may be interrupted with one or more hetero atoms chosen from an oxygen, sulphur or nitrogen atom, and which may be substituted with one or more hydroxyl, C₁-C₆ alkoxy or amino radicals, and which may bear one or more ketone functions;

the ring members E, G, J, L and M, which may be identical or different, represent a carbon, oxygen, sulphur or nitrogen atom and form a ring chosen from pyridine, pyrimidine, pyrazine, triazine and pyridazine rings;

p is an integer between 0 and 3 inclusive;

m is an integer between 0 and 5 inclusive;

p+m is an integer between 0 and 5;

the radicals R₁₁, which may be identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical substituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₁₁ are borne by a carbon atom,

the radicals R₁₂, which may be identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical or a benzyl radical; it being understood that the radicals R₁₂ are borne by a nitrogen atom,

R₁₃ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;

x is 0 or 1

when x=0, the linker arm D is attached to the nitrogen atom,

when x=1, the linker arm D is attached to one of the ring members E, G, J, L or M,

Y is a counterion.

Preferably, the ring members E, G, J, L and M form with the ring nitrogen a pyridine or pyrimidine ring.

When x is equal to 0, then R₁₁ is preferably chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical mono- or disubstituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical, and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical or a C₁-C₆ carbamylalkyl radical.

When x is equal to 1, R₁₃ is preferably chosen from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₁₁ is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical mono- or disubstituted with a (C₁-C₆)alkyl, (C₁-C₆)alkylcarbonyl, amido or (C₁-C₆)alkylsulphonyl radical; and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical and a C₁-C₆ carbamylalkyl radical.

Preferably, R₁₁, R₁₂ and R₁₃ are alkyl radicals that may be substituted.

The radical R₂ may also represent an onium radical of formula

—XP(O)(O—)OCH₂CH₂N⁺(CH₃)₃

in which X represents an oxygen atom or a radical —NR₁₄, R₁₄ representing a hydrogen, a C₁-C₄ alkyl radical or a hydroxyalkyl radical.

In the context of the invention, R₂ may also represent a guanidine radical of formula —X—C═NR₈—NR₉R₁₀, X represents an oxygen atom or a radical —NR₁₁, R₈, R₉, R₁₀ and R₁₁ representing a hydrogen, a C₁-C₄ alkyl radical or a hydroxyalkyl radical. According to one particular embodiment, X is —NR₁₁, R₈ is a hydrogen and R₉ and R₁₀ are chosen from hydrogen and an alkyl radical, preferably methyl.

The pKa of the guanidine radical R₂ is generally such that this substituent is present in cationic form (═NR₈H⁺) under the standard conditions for the oxidation dyeing of hair.

In the context of the invention, the counterion may be derived from a halogen atom such as bromine, chlorine, fluorine or iodine, a hydroxide, a citrate, a succinate, a tartrate, a lactate, a tosylate, a mesylate, a benzenesulphonate, an acetate, a hydrogen sulphate or a C₁-C₆ alkyl sulphate such as, for example, methyl sulphate or ethyl sulphate.

In the context of the present patent application, the cationic tertiary para-phenylenediamines containing a pyrrolidine nucleus described above, for which R₂ is of formula II or III, are preferably used. Even more preferably, the cationic tertiary para-phenylenediamines containing a pyrrolidine nucleus described above, for which R₂ is of formula II or III, with x=0 and for which n=0, are used.

Examples of derivatives of formula (I) that may be mentioned include:

| Formula | Nomenclature |
|---|---|
| 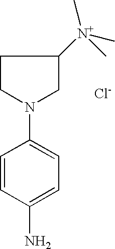 | [1-(4- Aminophenyl) pyrrolidin-3- yl-trimethyl- ammonium chloride (1) |
| 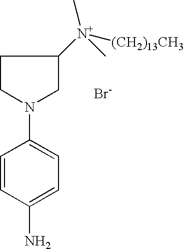 | Aminophenyl)py rrolidin-3- yl]dimethyltetra decyl- ammonium bromide (2) |
| 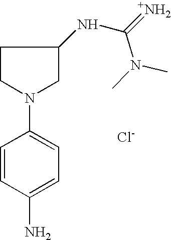 | N′-[1-(4- Aminophenyl) pyrrolidin-3- yl]-N,N- dimethyl guanidinium chloride (3) |
| 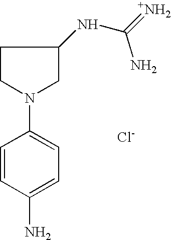 | N′-[1-(4- Aminophenyl)- pyrrolidin-3- yl]guanidinium chloride (4) |
| 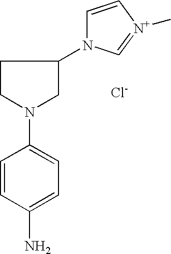 | 3-[1-(4- Aminophenyl) pyrrolidin-3- yl]-1-methyl- 3H- imidazole-1- ium chloride (5) |
| 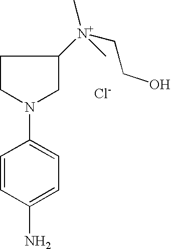 | [1-(4- Aminophenyl)py rrolidin-3-yl]- (2-hydroxy- ethyl)dimethyl- ammonium chloride (6) |
| 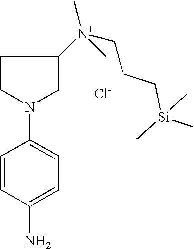 | [1-(4- Aminophenyl) pyrrolidin-3- yl]dimethyl- (3- trimethylsilan ylpropyl)amm onium chloride (7) |
| 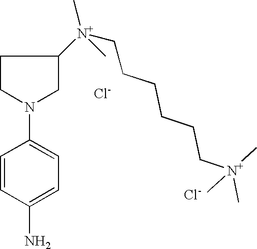 [1-(4- Aminophenyl)py rrolidin-3-yl]- (trimethyl- ammoniumhexyl )dimethyl- ammonium dichloride (8) | |
| 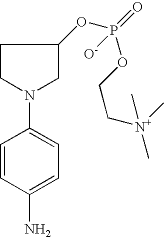 | [1-(4- Aminophenyl) -pyrrolidin-3- yl]oxophosph oryl choline (9) |
| 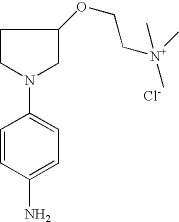 | {2-[1-(4- Aminophenyl)- pyrrolidin-3- yloxy]-ethyl}- trimethyl- ammonium chloride (10) |
| 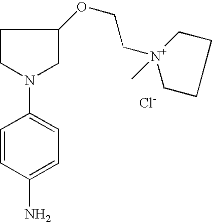 | 1-{2-[1-(4- Aminophenyl) pyrrolidin-3- yloxy]ethyl}- 1-methyl- pyrrolidinium; chloride (11) |
| 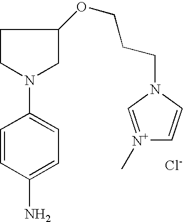 | 3-{3-[1-(4- Aminophenyl)py rrolidin-3- yloxy]-propyl}- 1-methyl-3H- imidazol-1-ium; chloride (12) |
| 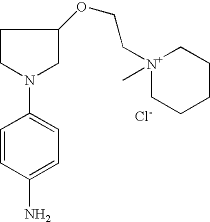 | 1-{2-[1-(4- Aminophenyl) pyrrolidin-3- yloxy]ethyl}- 1-methyl piperidinium; chloride (13) |
| 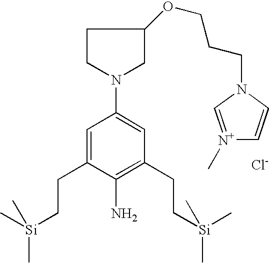 | 3-{3-[1-(5- trimethylsilanyle thyl-4-Amino-3- trimethylsilanyl- ethylphenyl)- pyrrolidin-3- yloxy]propyl}-1- methyl-3H- imidazole-1- um; chloride (14) |
| 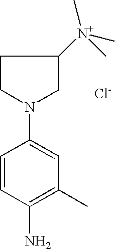 | [1-(4-Amino- 3- methylphenyl )pyrrolidin-3- yl]- trimethylamm oniumchloride (15) |
| 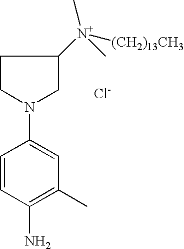 | [1-(4-Amino-3- methylphenyl)- pyrrolidin-3- yl]dimethyl- tetradecyl- ammonium chloride (16) |
| 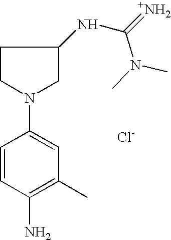 | N′-[1-(4- Amino-3- methylphenyl )-pyrrolidin- 3-yl]-N,N- dimethyl guanidinium chloride (17) |
| 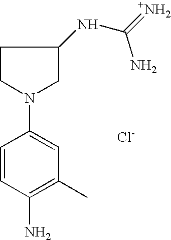 | N-[1-(4-Amino- methylphenyl)- pyrrolidin-3-yl]- guanidinium chloride (18) |
| 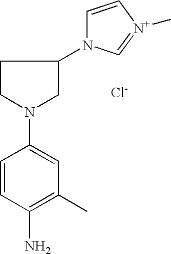 | 3-[1-(4- Amino-3- methylphenyl )-pyrrolidin- 3-yl]-1- methyl-3H- imidazole-1- ium chloride (19) |
| 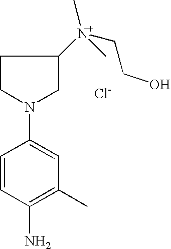 | [1-(4-Amino-3- methylphenyl)- pyrrolidin-3-yl]- (2- hydroxyethyl)- dimethyl- ammonium chloride (20) |
| 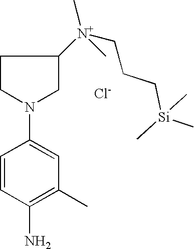 | [1-(4-Amino- 3- methylphenyl )pyrrolidin-3- yl]- dimethyl(3- trimethylsilan yl- propylammon ium chloride (21) |
| 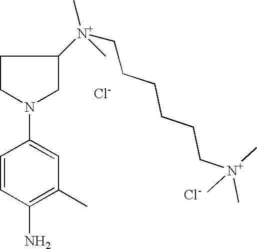 | [1-(4-Amino-3- methylphenyl)- pyrrolidin-3-yl]- (trimethyl- ammonium- hexyl)dimethyl- ammonium dichloride (22) |
| 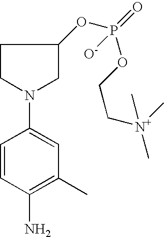 | [1-(4-Amino- 3- methylphenyl )-pyrrolidin- 3-yl]- oxophosphory lcholine (23) |
| 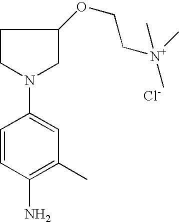 | {2-[1-(4-Amino- 3- methylphenyl)- pyrrolidin-3- yloxy]ethyl}- trimethyl- ammonium chloride (24) |
| 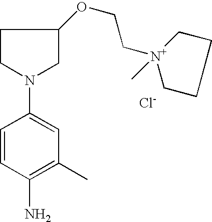 | 1-{2-[1-(4- Amino-3- methylphenyl )pyrrolidin-3- yloxy]ethyl}- 1-methyl- pyrrolidinium chloride (25) |
| 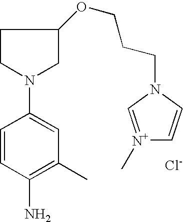 | 3-{3-[1-(4- Amino-3- methylphenyl)- pyrrolidin-3- yloxy]propyl}-1- methyl-3H- imidazole-1-um chloride (26) |
| 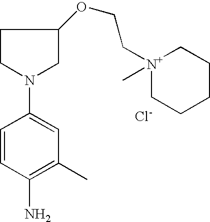 | 1-{2-[1-(4- Amino-3- methylphenyl )pyrrolidin-3- yloxy]ethyl}- 1- methylpiperid inium chloride (27) |
| 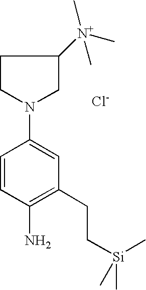 | [1-(4-Amino-3- trimethylsilanyle thylphenyl)- pyrrolidin-3-yl]- trimethyl- ammonium chloride (28) |
| 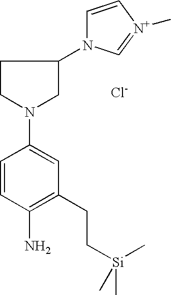 | 3-[1-(4- Amino-3- trimethylsilan ylethyl- phenyl)pyrrol idin-3-yl]-1- methyl-3H- imidazole-1- ium chloride (29) |
| 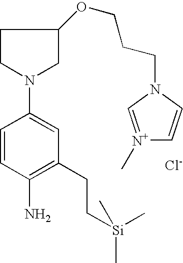 | 3-{3-[1-(4- Amino-3- trimethylsilanyle thylphenyl)- pyrrolidin-3- yloxy]propyl}-1- methyl-3H- imidazole-1-um chloride (30) |
| 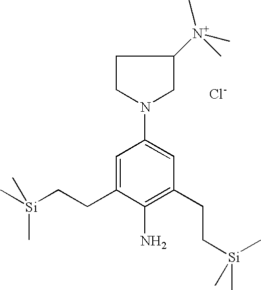 | [1-(5- trimethylsilan ylethyl-4- Amino-3- trimethylsilan ylethylphenyl )pyrrolidin-3- yl]- trimethylamm oniumchloride (31) |
| 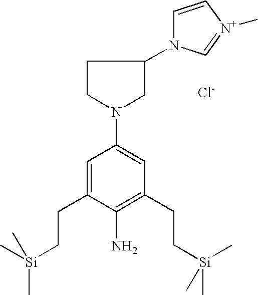 | 3-[1-(5- trimethylsilanyle thyl-4-Amino-3- trimethylsilanyle thylphenyl)- pyrrolidin-3-yl]- 1-methyl-3H- imidazole-1-ium chloride (32) |
| 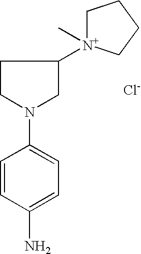 | 1′-(4- Aminophenyl) -1-methyl- [1,3′]bipyrroli dinyl-1-ium chloride (33) |
| 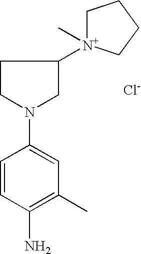 | 1′-(4-Amino-3- methylphenyl)- 1-methyl- [1,3′]bipyrrolidi nyl-1-ium chloride (34) |
| 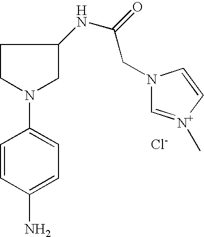 | 3-{[1-(4- Aminophenyl) pyrrolidin-3- ylcarbamoyl]- methyl}-1- methyl-3H- imidazole-1- ium chloride (35) |
| 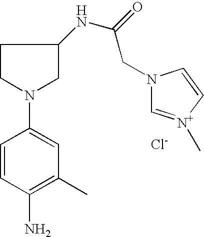 | 3-{[1-(4-Amino- 3- methylphenyl)- pyrrolidin-3- ylcarbamoyl]- methyl}-1- methyl-3H- imidazole-1-ium chloride (36) |
| 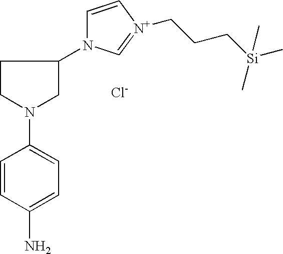 | 3-[1-(4- Aminophenyl) pyrrolidin-3- yl]-1-(3- trimethylsilan yl-propyl)- 3H- imidazole-1- ium chloride (37) |
| 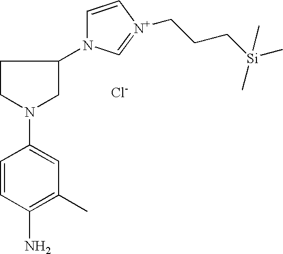 | 3-[1-(4- Aminophenyl)py rrolidin-3-yl]-1- (3- trimethylsilanyl- propyl)-3H- imidazole-1-ium chloride (38) |
| 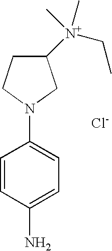 | [1-(4- Aminophenyl) pyrrolidin-3- yl]- ethyldimethyl ammonium chloride (39) |
| 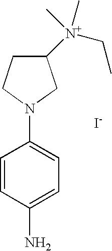 | [1-(4- Aminophenyl)py rrolidin-3-yl]- ethyldimethyl- ammonium iodide (40) |
| 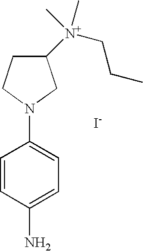 | [1-(4- aminophenyl) pyrrolidin-3- yl]- propyldimeth ylammonium iodide, (41) |
| 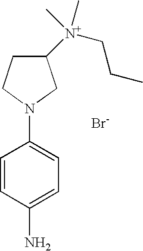 | [1-(4- Aminophenyl)py rrolidin-3-yl]- propyldimethyl- ammonium bromide (42) |
| 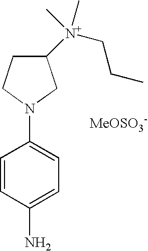 | [1-(4- Aminophenyl) pyrrolidin-3- yl]- propyldimeth ylammonium methosulphate (43) |
| 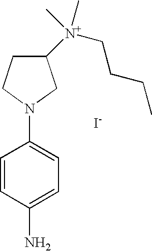 | [1-(4- Aminophenyl)py rrolidin-3-yl]- butyldimethyl- ammonium iodide (44) |
| 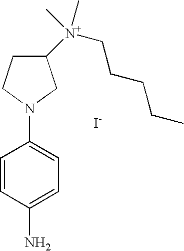 | [1-(4- Aminophenyl) pyrrolidin-3- yl]- pentyldimeth ylammonium iodide (45) |
| 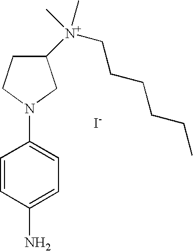 | [1-(4- Aminophenyl)py rrolidin-3-yl]- hexyldimethyl- ammonium iodide (46) |
| 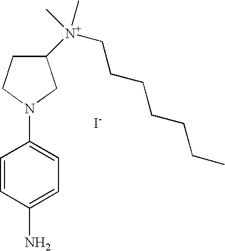 | [1-(4- Aminophenyl) pyrrolidin-3- yl]- heptyldimeth yl-ammonium iodide (47) |
| 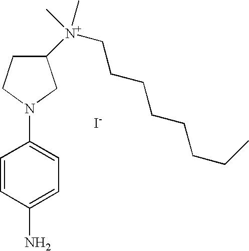 | [1-(4- Aminophenyl)py rrolidin-3-yl]- octyldimethyl- ammonium iodide (48) |
| 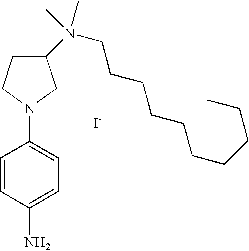 | [1-(4- Aminophenyl) pyrrolidin-3- yl]- decyldimethyl ammonium iodide (49) |
| 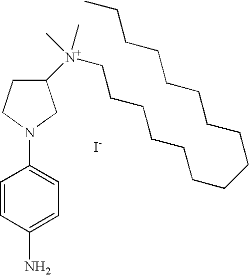 | [1-(4-amino- phenyl)pyrrolidi n-3-yl]- hexadecyldimeth ylammonium iodide (50) |
| 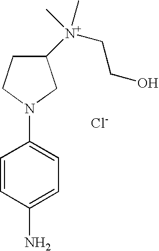 | [1-(4- Aminophenyl) pyrrolidin-3- yl]- hydroxyethyl- dimethylamm onium chloride (51) |
| 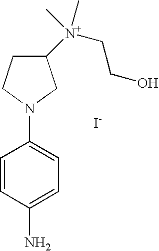 | [1-(4- aminophenyl)- pyrrolidin-3-yl]- hydroxyethyl- dimethyl- ammonium iodide (52) |

The derivatives of formula I that are preferably used are:

[1-(4-Aminophenyl)pyrrolidin-3-yl]trimethylammonium chloride;

[1-(4-Aminophenyl)pyrrolidin-3-yl]dimethyltetradecylammonium bromide;

N′-[1-(4-Aminophenyl)pyrrolidin-3-yl]-N,N-dimethyl guanidinium chloride;

N-[1-(4-Aminophenyl)pyrrolidin-3-yl]guanidinium chloride;

3-[1-(4-Aminophenyl)pyrrolidin-3-yl]-1-methyl-3H-imidazol-1-ium chloride;

[1-(4-Aminophenyl)pyrrolidin-3-yl](2-hydroxyethyl)dimethylammonium chloride;

[1-(4-Aminophenyl)pyrrolidin-3-yl]dimethyl(3-trimethylsilanylpropyl)ammonium chloride;

[1-(4-Amino-3-methylphenyl)pyrrolidin-3-yl]trimethylammonium chloride;

[1-(4-Amino-3-methylphenyl)pyrrolidin-3-yl]dimethyltetradecylammonium chloride;

N′-[1-(4-Amino-3-methylphenyl)pyrrolidin-3-yl]-N,N-dimethyl guanidinium chloride;

N-[1-(4-Amino-3-methylphenyl)pyrrolidin-3-yl]guanidinium chloride;

3-[1-(4-Amino-3-methylphenyl)pyrrolidin-3-yl]-1-methyl-3H-imidazol-1-ium chloride;

[1-(4-Amino-3-methylphenyl)pyrrolidin-3-yl](2-hydroxyethyl)dimethylammoniumchloride;

[1-(4-Amino-3-methylphenyl)pyrrolidin-3-yl]dimethyl(3-trimethylsilanylpropylammonium chloride;

1′-(4-Aminophenyl)-1-methyl[1,3′]bipyrrolidinyl-1-ium chloride;

1′-(4-Amino-3-methylphenyl)-1-methyl[1,3′]bipyrrolidinyl-1-ium chloride;

3-{[1-(4-Aminophenyl)pyrrolidin-3-ylcarbamoyl]methyl}-1-methyl-3H-imidazol-1-ium chloride;

3-{[1-(4-Amino-3-methylphenyl)pyrrolidin-3-ylcarbamoyl]methyl}-1-methyl-3H-imidazol-1-ium chloride;

3-[1-(4-Aminophenyl)pyrrolidin-3-yl]-1-(3-trimethylsilanylpropyl)-3H-imidazol-1-ium chloride;

3-[1-(4-Amino-3-methylphenyl)pyrrolidin-3-yl]-1-(3-trimethylsilanylpropyl)-3H-imidazol-1-ium chloride;

[1-(4-Aminophenyl)pyrrolidin-3-yl]ethyldimethylammonium chloride;

[1-(4-Aminophenyl)pyrrolidin-3-yl]ethyldimethylammonium iodide;

[1-(4-Aminophenyl)pyrrolidin-3-yl]propyldimethylammonium iodide;

[1-(4-Aminophenyl)pyrrolidin-3-yl]propyldimethylammonium bromide;

[1-(4-Aminophenyl)pyrrolidin-3-yl]propyldimethylammonium methosulphate;

[1-(4-Aminophenyl)pyrrolidin-3-yl]butyldimethylammonium iodide;

[1-(4-Aminophenyl)pyrrolidin-3-yl]pentyldimethylammonium iodide;

[1-(4-Aminophenyl)pyrrolidin-3-yl]hexyldimethylammonium iodide;

[1-(4-Aminophenyl)pyrrolidin-3-yl]heptyldimethylammonium iodide;

[1-(4-Aminophenyl)pyrrolidin-3-yl]octyldimethylammonium iodide;

[1-(4-Aminophenyl)pyrrolidin-3-yl]decyldimethylammonium iodide;

[1-(4-Aminophenyl)pyrrolidin-3-yl]hexadecyldimethylammonium iodide;

[1-(4-Aminophenyl)pyrrolidin-3-yl]hydroxyethyldimethylammonium chloride;

[1-(4-Aminophenyl)pyrrolidin-3-yl]hydroxyethyldimethylammonium iodide.

The following compounds will more preferably be used:

[1-(4-Aminophenyl)pyrrolidin-3-yl]trimethylammonium chloride;

[1-(4-Aminophenyl)pyrrolidin-3-yl]dimethyltetradecylammonium bromide;

N′-[1-(4-Aminophenyl)pyrrolidin-3-yl]-N,N-dimethyl guanidinium chloride;

N-[1-(4-Aminophenyl)pyrrolidin-3-yl]guanidinium chloride;

3-[1-(4-Aminophenyl)pyrrolidin-3-yl]-1-methyl-3H-imidazol-1-ium chloride;

[1-(4-Aminophenyl)pyrrolidin-3-yl](2-hydroxyethyl)dimethylammonium chloride;

[1-(4-Aminophenyl)pyrrolidin-3-yl]dimethyl(3-trimethylsilanylpropyl)ammonium chloride;

[1-(4-Aminophenyl)pyrrolidin-3-yl](trimethylammoniumhexyl)dimethylammonium dichloride;

1′-(4-Aminophenyl)-1-methyl[1,3′]bipyrrolidinyl-1-ium chloride;

3-[1-(4-Aminophenyl)pyrrolidin-3-yl]-1-(3-trimethylsilanylpropyl)-3H-imidazol-1-ium chloride;

3-[1-(4-Amino-3-methylphenyl)pyrrolidin-3-yl]-1-(3-trimethylsilanylpropyl)-3H-imidazol-1-ium chloride;

[1-(4-Aminophenyl)pyrrolidin-3-yl]ethyldimethylammonium chloride;

[1-(4-Aminophenyl)pyrrolidin-3-yl]ethyldimethylammonium iodide;

[1-(4-Aminophenyl)pyrrolidin-3-yl]propyldimethylammonium iodide;

[1-(4-Aminophenyl)pyrrolidin-3-yl]propyldimethylammonium bromide;

[1-(4-Aminophenyl)pyrrolidin-3-yl]propyldimethylammonium methosulphate;

[1-(4-Aminophenyl)pyrrolidin-3-yl]butyldimethylammonium iodide;

[1-(4-Aminophenyl)pyrrolidin-3-yl]pentyldimethylammonium iodide;

[1-(4-Aminophenyl)pyrrolidin-3-yl]hexyldimethylammonium iodide;

[1-(4-Aminophenyl)pyrrolidin-3-yl]heptyldimethylammonium iodide;

[1-(4-Aminophenyl)pyrrolidin-3-yl]octyldimethylammonium iodide;

[1-(4-Aminophenyl)pyrrolidin-3-yl]decyldimethylammonium iodide;

[1-(4-Aminophenyl)pyrrolidin-3-yl]hexadecyldimethylammonium iodide;

[1-(4-Aminophenyl)pyrrolidin-3-yl]hydroxyethyldimethylammonium chloride;

[1-(4-Aminophenyl)pyrrolidin-3-yl]hydroxyethyldimethylammonium iodide.

Even more preferably, the following compounds will be used:

[1-(4-Aminophenyl)pyrrolidin-3-yl]trimethylammonium chloride;

3-[1-(4-Aminophenyl)pyrrolidin-3-yl]-1-methyl-3H-imidazol-1-ium chloride;

[1-(4-Aminophenyl)pyrrolidin-3-yl](2-hydroxyethyl)dimethylammonium chloride;

1′-(4-Aminophenyl)-1-methyl[1,3′]bipyrrolidinyl-1-ium chloride; and most particularly

[1-(4-Aminophenyl)pyrrolidin-3-yl]trimethylammonium chloride; and

[1-(4-Aminophenyl)pyrrolidin-3-yl](2-hydroxyethyl)dimethylammonium chloride.

The counterion is not critical as regards the result of the invention; any compound similar to the preferred compounds described above, but with a different counterion, forms an integral part of the preferred compounds.

The cationic tertiary para-phenylenediamine(s) containing a pyrrolidine nucleus represent from 0.001% to 10% and preferably from 0.005% to 6% by weight relative to the total weight of the composition.

The compounds of formula (I) may be synthesized according to known methods, described especially in patent application WO 02/45675.

The polyol esters that may be used as essential agents in the compositions of the invention are in particular esters of saturated or unsaturated, linear or branched C₂-C₃₀ acid, whose hydrocarbon-based chain is optionally substituted with one or more hydroxyl groups, and of optionally ethoxylated, linear, branched or cyclic C₂-C₅₀ polyols.

Among the acids that are preferred, mention may be made of lauric acid, stearic acid, isostearic acid, oleic acid and ricinoleic acid.

Among the polyols that may be mentioned are ethylene glycol, propylene glycol, glycerol, glucose, sorbitol and sorbitol anhydride.

Among the polyol esters that may be used in the compositions according to the invention, mention may be made of ethylene glycol monostearate or distearate (for example Cutina AGS from Cognis or Tegin EGS from Goldschmidt), glyceryl oleate or stearate (for example Stepan GMO from Stepan, Stepan GML from Stepan and Stepan GMS from Stepan), and polyethylene glycol laurates, oleates and stearates (for example Stepan PEG 400 ML from Stepan, Stepan PEG 200 MO from Stepan and Stepan PEG MS from Stepan).

Ethoxylated polyol esters such as polyoxyethylenated ethers of glyceryl laurates, oleates or stearates, and polyoxyethylenated ethers of sorbitol laurates and/or of sorbitol anhydride (polysorbates and especially Polysorbate 20) are preferred.

The number of ethylene oxides in these ethers is then between 1 and 200 and preferably from 2 to 100 (for example AEC PEG 12-glyceryl laurates from A and B Connock, Nikkol TMGO-10 from Nikko, AEC PEG-5-Glyceryl Stearate from A and E Connock, and AEC Polysorbate 20 from A and E Connock).

The polyol ester(s) is (are) present in concentrations ranging from 0.01% to 20%, preferably from 0.05% to 10% and even more preferably from 0.1% to 5% relative to the total weight of the composition.

According to a first preferred mode, the composition according to the present invention also contains at least one cationic polymer.

For the purposes of the present invention, the term “cationic polymer” denotes any polymer containing cationic groups and/or groups that may be ionized into cationic groups.

The cationic polymers that may be used in accordance with the present invention may be chosen from all those already known per se as improving the cosmetic properties of the hair, i.e. especially those described in patent application EP-A-337 354 and in French patents FR-2 270 846, 2 383 660, 2 598 611, 2 470 596 and 2 519 863.

The cationic polymers that are preferred are chosen from those containing units comprising primary, secondary, tertiary and/or quaternary amine groups, which may either form part of the main polymer chain or may be borne by a side substituent directly attached thereto.

The cationic polymers used generally have a number-average molecular mass of between 500 and 5×10⁶ approximately and preferably between 10³ and 3×10⁶ approximately.

Among the cationic polymers that may be mentioned more particularly are polymers of the polyamine, polyamino amide and polyquaternary ammonium type.

These are known products. They are described in particular in French patents Nos 2 505 348 and 2 542 997. Among the said polymers, mention may be made of:

(1) Homopolymers or copolymers derived from acrylic or methacrylic esters or amides and comprising at least one of the units of formula (VI), (VII), (VIII) or (IX) below:

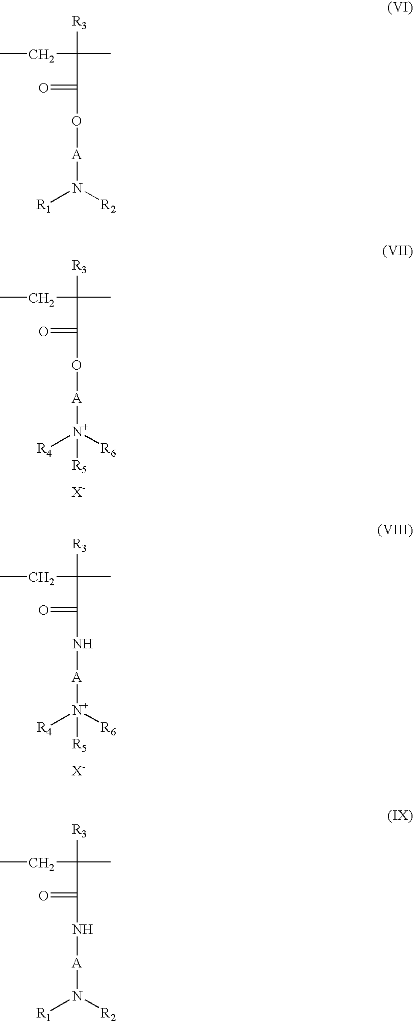

in which:

R₃ denotes a hydrogen atom or a CH₃ radical;

A represents a linear or branched alkyl group containing 1 to 6 carbon atoms, preferably 2 or 3 carbon atoms, or a hydroxyalkyl group containing 1 to 4 carbon atoms;

R₄, R₅ and R₆, which may be identical or different, represent an alkyl group containing from 1 to 18 carbon atoms or a benzyl radical and preferably an alkyl group containing from 1 to 6 carbon atoms;

R₁ and R₂, which may be identical or different, represent hydrogen or an alkyl group containing from 1 to 6 carbon atoms, and preferably methyl or ethyl;

X⁻ denotes an anion derived from an inorganic or organic acid, such as a methosulphate anion or a halide such as chloride or bromide.

The polymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these polymers of family (1), mention may be made of:

copolymers of acrylamide and of dimethylaminoethyl methacrylate quaternized with dimethyl sulphate or with a dimethyl halide, such as the product sold under the name Hercofloc by the company Hercules,

the copolymers of acrylamide and of methacryloyloxyethyltrimethylammonium chloride described, for example, in patent application EP-A-080 976 and sold under the name Bina Quat P 100 by the company Ciba Geigy,

the copolymer of acrylamide and of methacryloyloxyethyltrimethylammonium methosulphate sold under the name Reten by the company Hercules,

quaternized or non-quaternized vinylpyrrolidone/dialkylaminoalkyl acrylate or methacrylate copolymers, such as the products sold under the name “Gafquat” by the company ISP, such as, for example, “Gafquat 734” or “Gafquat 755”, or alternatively the products known as “Copolymer 845, 958 and 937”. These polymers are described in detail in French patents 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold in particular under the name Styleze CC 10 by ISP, and

quaternized vinylpyrrolidone/dimethylaminopropyl methacrylamide copolymers such as the product sold under the name “Gafquat HS 100” by the company ISP.

(2) The cellulose ether derivatives containing quaternary ammonium groups, described in French patent 1 492 597, and in particular polymers sold under the name “JR” (JR 400, JR 125 and JR 30M) or “LR” (LR 400 or LR 30M) by the company Union Carbide Corporation. These polymers are also defined in the CTFA dictionary as quaternary ammoniums of hydroxyethylcellulose that have reacted with an epoxide substituted with a trimethylammonium group.

(3) Cationic cellulose derivatives such as cellulose copolymers or cellulose derivatives grafted with a water-soluble quaternary ammonium monomer, and described in particular in U.S. Pat. No. 4,131,576, such as hydroxyalkylcelluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt.

The commercial products corresponding to this definition are more particularly the products sold under the names “Celquat L 200” and “Celquat H 100” by the company National Starch.

(4) The cationic polysaccharides described more particularly in U.S. Pat. Nos. 3,589,578 and 4,031,307, such as guar gums containing cationic trialkylammonium groups. Guar gums modified with a salt (e.g. chloride) of 2,3-epoxypropyltrimethylammonium are used, for example.

Such products are sold in particular under the trade names Jaguar C13 S, Jaguar C 15, Jaguar C 17 and Jaguar C162 by the company Meyhall.

(5) Polymers consisting of piperazinyl units and of divalent alkylene or hydroxyalkylene radicals containing straight or branched chains, optionally interrupted with oxygen, sulphur or nitrogen atoms or by aromatic or heterocyclic rings, as well as the oxidation and/or quaternization products of these polymers. Such polymers are described, in particular, in French patents 2 162 025 and 2 280 361.

(6) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508.

(7) The polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical contains from 1 to 4 carbon atoms and preferably denotes methyl, ethyl or propyl. Such polymers are described in particular in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(8) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(9) Cyclopolymers of alkyldiallylamine or of dialkyldiallylammonium, such as the homopolymers or copolymers containing, as main constituent of the chain, units corresponding to formula (X) or (XI):

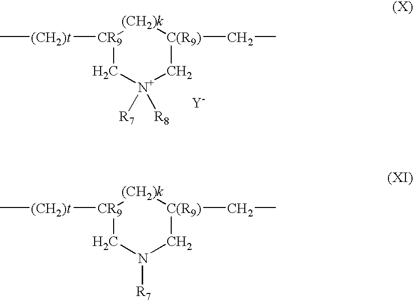

in which formulae k and t are equal to 0 or 1, the sum k+t being equal to 1; R₉ denotes a hydrogen atom or a methyl radical; R₇ and R₈, independently of each other, denote an alkyl group having from 1 to 6 carbon atoms, a hydroxyalkyl group in which the alkyl group preferably has 1 to 5 carbon atoms, a lower (C₁-C₄) amidoalkyl group, or R₇ and R₈ can denote, together with the nitrogen atom to which they are attached, heterocyclic groups such as piperidyl or morpholinyl; R₇ and R₈, independently of each other, preferably denote an alkyl group containing from 1 to 4 carbon atoms; Y⁻ is an anion such as bromide, chloride, acetate, borate, citrate, tartrate, bisulphate, bisulphite, sulphate or phosphate. These polymers are described in particular in French patent 2 080 759 and in its Certificate of Addition 2 190 406.

Among the polymers defined above, mention may be made more particularly of the dimethyldiallylammonium chloride homopolymer sold under the name Merquat 100 by the company Calgon (and its homologs of low weight-average molecular mass) and the copolymers of diallyldimethylammonium chloride and of acrylamide sold under the name Merquat 550.

(10) The quaternary diammonium polymer containing repeating units corresponding to the formula:

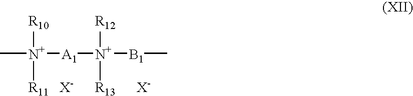

in which formula (XII):

R₁₀, R₁₁, R₁₂ and R₁₃, which may be identical or different, represent aliphatic, alicyclic or arylaliphatic radicals containing from 1 to 20 carbon atoms or lower hydroxyalkylaliphatic radicals, or alternatively R₁₀, R₁₁, R₁₂ and R₁₃, together or separately, constitute, with the nitrogen atoms to which they are attached, heterocycles optionally containing a second hetero atom other than nitrogen, or alternatively R₁₀, R₁₁, R₁₂ and R₁₃ represent a linear or branched C₁-C₆ alkyl radical substituted with a nitrile, ester, acyl or amide group or a group —CO—O—R₁₄—D or —CO—NH—R₁₄—D where R₁₄ is an alkylene and D is a quaternary ammonium group;

A₁ and B₁ represent polymethylene groups containing from 2 to 20 carbon atoms which may be linear or branched, saturated or unsaturated, and which may contain, linked to or intercalated in the main chain, one or more aromatic rings or one or more oxygen or sulphur atoms or sulphoxide, sulphone, disulphide, amino, alkylamino, hydroxyl, quaternary ammonium, ureido, amide or ester groups, and

X⁻ denotes an anion derived from a mineral or organic acid;

A₁, R₁₀ and R₁₂ can form, with the two nitrogen atoms to which they are attached, a piperazine ring; in addition, if A₁ denotes a linear or branched, saturated or unsaturated alkylene or hydroxyalkylene radical, B₁ can also denote a group —(CH₂)ₙ—CO—D—OC—(CH₂)ₙ— in which D denotes:

a) a glycol residue of formula: —O—Z—O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;

d) a ureylene group of formula: —NH—CO—NH—.

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to formula (XIII) below:

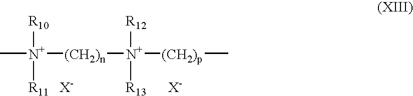

in which R₁₀, R₁₁, R₁₂ and R₁₃, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

(11) Polyquaternary ammonium polymers consisting of repeating units of formula (XIV):

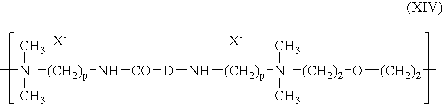

in which p denotes an integer ranging from 1 to 6 approximately, D may be nothing or may represent a group —(CH₂)ᵣ—CO— in which r denotes a number equal to 4 or 7, and X⁻ is an anion.

Such polymers may be prepared according to the processes described in U.S. Pat. Nos. 4,157,388, 4,702,906 and 4,719,282. They are especially described in patent application EP-A-122 324.

Among these products, mention may be made, for example, of Mirapol A 15, Mirapol AD1, Mirapol AZ1 and Mirapol 175 sold by the company Miranol.

(12) Quaternary polymers of vinylpyrrolidone and of vinylimidazole, such as, for example, the products sold under the names Luviquat FC 905, FC 550 and FC 370 by the company BASF.

(13) Polyamines such as Polyquart H sold by Henkel, which is given under the reference name “Polyethylene glycol (15) tallow polyamine” in the CTFA dictionary.

(14) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more particularly. This dispersion is sold under the name Salcare® SC 92 by the company Allied Colloids. A crosslinked methacryloyloxyethyltrimethylammonium chloride homopolymer containing about 50% by weight of the homopolymer in mineral oil or in a liquid ester can also be used. These dispersions are sold under the names Salcare® SC 95 and Salcare® SC 96 by the company Allied Colloids.

Other cationic polymers that can be used in the context of the invention are polyalkyleneimines, in particular polyethyleneimines, polymers containing vinylpyridine or vinylpyridinium units, condensates of polyamines and of epichlorohydrin, quaternary polyureylenes and chitin derivatives.

Among all the cationic polymers that may be used in the context of the present invention, it is preferred to use the polymers of families (1), (9), (10), (11) and (14) and even more preferably the polymers containing repeating units of formulae (W) and (U) below:

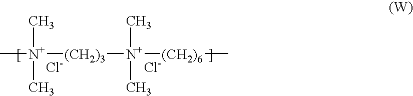

and in particular those whose molecular weight, determined by gel permeation chromatography, is between 9500 and 9900;

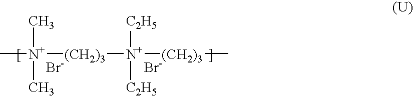

and especially those whose molecular weight, determined by gel permeation chromatography, is about 1200.

The concentration of cationic polymer in the composition according to the present invention may range from 0.01% to 10%, preferably from 0.05% to 5% and even more preferably from 0.1% to 3% by weight relative to the total weight of the composition.

According to a second preferred mode, the composition according to the present invention also contains at least one thickening polymer, also known as a “rheology modifier”.

The rheology modifiers may be chosen from fatty acid amides (coconut diethanolamide or monoethanolamide, or oxyethylenated monoethanolamide of carboxylic acid alkyl ether), cellulose-based thickeners (hydroxyethylcellulose, hydroxypropylcellulose or carboxymethylcellulose), guar gum and its derivatives (hydroxypropylguar), gums of microbial origin (xanthan gum, scleroglucan gum), crosslinked homopolymers of acrylic acid or of acrylamidopropanesulphonic acid, and associative polymers as described below.

The associative polymers that may be used according to the invention are water-soluble polymers capable, in an aqueous medium, of reversibly combining with each other or with other molecules.

Their chemical structure comprises hydrophilic zones and hydrophobic zones characterized by at least one fatty chain.

The associative polymers that may be used according to the invention may be of anionic, cationic, amphoteric or, preferably, nonionic type.

Their weight concentration in the dye composition may range from about 0.01% to 10% of the total weight of the composition, and in the ready-to-use composition (comprising the oxidizing agent), from about 0.0025% to 10% of the total weight of the composition. More preferably, this amount ranges from about 0.1% to 5% by weight in the dye composition and from about 0.025% to 10% in the ready-to-use composition.

Among the associative polymers of anionic type that may be mentioned are:

(I) those comprising at least one hydrophilic unit and at least one fatty-chain allyl ether unit, more particularly those whose hydrophilic unit consists of an ethylenic unsaturated anionic monomer, more particularly of a vinylcarboxylic acid and most particularly of an acrylic acid or a methacrylic acid or mixtures thereof, the fatty-chain allyl ether unit of which corresponds to the monomer of formula (XV) below:

CH₂═CR′CH₂OBₙR (XV)

in which R′ denotes H or CH₃, B denotes an ethyleneoxy radical, n is zero or denotes an integer ranging from 1 to 100, R denotes a hydrocarbon-based radical chosen from alkyl, arylalkyl, aryl, alkylaryl and cycloalkyl radicals, containing from 8 to 30 carbon atoms, preferably 10 to 24 carbon atoms and even more particularly from 12 to 18 carbon atoms. A unit of formula (XV) that is more particularly preferred is a unit in which R′ denotes H, n is equal to 10 and R denotes a stearyl (C₁₈) radical.

Anionic associative polymers of this type are described and prepared, according to an emulsion polymerization process, in patent EP-0 216 479.

Among these anionic associative polymers that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether of formula (XV), and from 0% to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), in particular those sold by the company Allied Colloids under the names Salcare SC 80® and Salcare SC 90®, which are aqueous 30% emulsions of a crosslinked terpolymer of methacrylic acid, of ethyl acrylate and of steareth-10 allyl ether (40/50/10).

(II) those comprising at least one hydrophilic unit of unsaturated olefinic carboxylic acid type, and at least one hydrophobic unit of (C₁₀-C₃₀)alkyl ester of unsaturated carboxylic acid type.

Preferably, these polymers are chosen from those in which the hydrophilic unit of unsaturated olefinic carboxylic acid type corresponds to the monomer of formula (XVI) below:

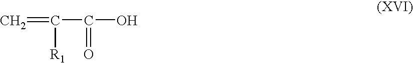

in which R₁ denotes H or CH₃ or C₂H₅, that is to say acrylic acid, methacrylic acid or ethacrylic acid units, and in which the hydrophobic unit of (C₁₀-C₃₀)alkyl ester of unsaturated carboxylic acid type corresponds to the monomer of formula (XVII) below:

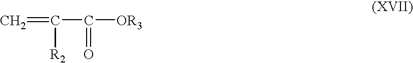

in which R₂ denotes H or CH₃ or C₂H₅ (that is to say acrylate, methacrylate or ethacrylate units) and preferably H (acrylate units) or CH₃ (methacrylate units), R₃ denoting a C₁₀-C₃₀ and preferably C₁₂-C₂₂ alkyl radical.

(C₁₀-C₃₀) alkyl esters of unsaturated carboxylic acids according to the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. Nos. 3,915,921 and 4,509,949.

Among the anionic associative polymers of this type that will be used more particularly are polymers formed from a monomer mixture comprising:

(i) essentially acrylic acid,

(ii) an ester of formula (XVII) described above in which R₂ denotes H or CH₃, R₃ denoting an alkyl radical containing from 12 to 22 carbon atoms, and

(iii) a crosslinking agent, which is a well-known copolymerizable unsaturated polyethylenic monomer, for instance diallyl phthalate, allyl (meth)acrylate, divinylbenzene, (poly)ethylene glycol dimethacrylate and methylenebisacrylamide.

Among anionic associative polymers of this type that will be used more particularly are those consisting of from 95% to 60% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among the said above polymers, those most particularly preferred according to the present invention are the products sold by the company Goodrich under the trade names Pemulen TR1®, Pemulen TR2® and Carbopol 1382®, and even more preferentially Pemulen TR1®, and the product sold by the company SEPPIC under the name Coatex SX®.

(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.

(IV) acrylic terpolymers comprising:

(a) about 20% to 70% by weight of a carboxylic acid containing α,β-monoethylenic unsaturation,

(b) about 20% to 80% by weight of a non-surfactant monomer containing α,β-monoethylenic unsaturation other than (a),

(c) about 0.5% to 60% by weight of a nonionic monourethane which is the product of reaction of a monohydric surfactant with a monoisocyanate containing monoethylenic unsaturation,

such as those described in patent application EP-A-0 173 109 and more particularly the terpolymer described in Example 3, namely a methacrylic acid/methyl acrylate/behenyl dimethyl-meta-isopropenylbenzylisocyanate ethoxylated (40 EO) terpolymer, as an aqueous 25% dispersion.

(V) copolymers comprising among their monomers a carboxylic acid containing α,β-monoethylenic unsaturation and an ester of a carboxylic acid containing α,β-monoethylenic unsaturation and of an oxyalkylenated fatty alcohol.

Preferentially, these compounds also comprise as monomer an ester of a carboxylic acid containing α,β-monoethylenic unsaturation and of a C₁-C₄ alcohol.

An example of a compound of this type that may be mentioned is Aculyn 22® sold by the company Rohm & Haas, which is a methacrylic acid/ethyl acrylate/stearyl methacrylate oxyalkylenated terpolymer.

Among the associative polymers of cationic type that may be mentioned are:

(I) cationic associative polyurethanes, the family of which has been described by the Applicant in French patent application No. 00/09609; it may be represented by the general formula (XVIII) below:

R—X—(P)ₙ—[L—(Y)ₘ]ᵣ—L′—(P′)ₚ—X′—R′ (XVIII)

in which:

R and R′, which may be identical or different, represent a hydrophobic group or a hydrogen atom;

X and X′, which may be identical or different, represent a group comprising an amine function optionally bearing a hydrophobic group, or alternatively a group L″;

L, L′ and L″, which may be identical or different, represent a group derived from a diisocyanate;

P and P′, which may be identical or different, represent a group comprising an amine function optionally bearing a hydrophobic group;

Y represents a hydrophilic group;

r is an integer between 1 and 100, preferably between 1 and 50 and in particular between 1 and 25;

n, m and p each range, independently of each other, from 0 to 1000;

the molecule containing at least one protonated or quaternized amine function and at least one hydrophobic group.

In one preferred embodiment of these polyurethanes, the only hydrophobic groups are the groups R and R′ at the chain ends.

One preferred family of cationic associative polyurethanes is the one corresponding to formula (XVIII) described above and in which:

R and R′ both independently represent a hydrophobic group,

X and X′ each represent a group L″,

n and p are between 1 and 1000, and

L, L′, L″, P, P′, Y and m have the meaning given above.

Another preferred family of cationic associative polyurethanes is the one corresponding to formula (XVIII) above in which:

R and R′ both independently represent a hydrophobic group, X and X′ each represent a group L″, n and p are 0, and L, L′, L″, Y and m have the meaning given above.

The fact that n and p are 0 means that these polymers do not comprise units derived from a monomer containing an amine function, incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide, a sulphate, etc.

Yet another preferred family of cationic associative polyurethanes is the one corresponding to formula (XVIII) above in which:

R and R′ both independently represent a hydrophobic group,

X and X′ both independently represent a group comprising a quaternary amine,

n and p are zero, and

L, L′, Y and m have the meaning given above.

The number-average molecular mass of the cationic associative polyurethanes is preferably between 400 and 500 000, in particular between 1000 and 400 000 and ideally between 1000 and 300 000.

The expression “hydrophobic group” means a radical or polymer containing a saturated or unsaturated, linear or branched hydrocarbon-based chain, which may contain one or more hetero atoms such as P, O, N or S, or a radical containing a perfluoro or silicone chain. When the hydrophobic group denotes a hydrocarbon-based radical, it comprises at least 10 carbon atoms, preferably from 10 to 30 carbon atoms, in particular from 12 to 30 carbon atoms and more preferably from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer such as, for example, polybutadiene.

When X and/or X′ denote(s) a group comprising a tertiary or quaternary amine, X and/or X′ may represent one of the following formulae:

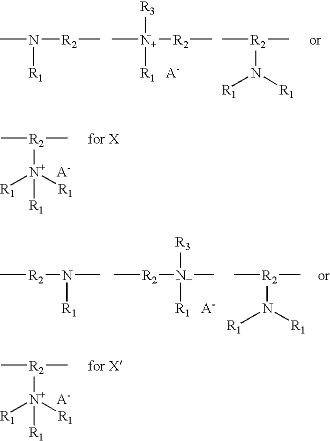

in which:

R₂ represents a linear or branched alkylene radical containing from 1 to 20 carbon atoms, optionally comprising a saturated or unsaturated ring, or an arylene radical, one or more of the carbon atoms possibly being replaced with a hetero atom chosen from N, S, O and P;

R₁ and R₃, which may be identical or different, denote a linear or branched C₁-C₃₀ alkyl or alkenyl radical or an aryl radical, at least one of the carbon atoms possibly being replaced with a hetero atom chosen from N, S, O and P;

A⁻ is a physiologically acceptable counterion.

The groups L, L′ and L″ represent a group of formula:

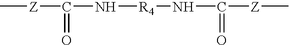

in which:

Z represents —O—, —S— or —NH—; and

R₄ represents a linear or branched alkylene radical containing from 1 to 20 carbon atoms, optionally comprising a saturated or unsaturated ring, or an arylene radical, one or more of the carbon atoms possibly being replaced with a hetero atom chosen from N, S, O and P.

The groups P and P′ comprising an amine function may represent at least one of the following formulae:

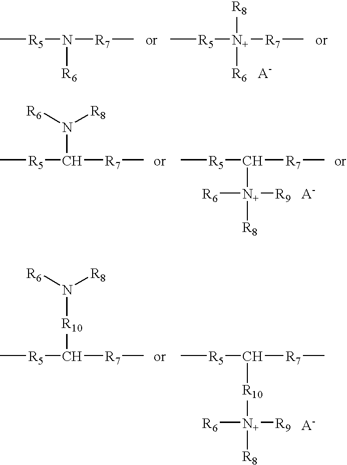

in which:

R₅ and R₇ have the same meanings as R₂ defined above;

R₆, R₈ and R₉ have the same meanings as R₁ and R₃ defined above;

R₁₀ represents a linear or branched, optionally unsaturated alkylene group possibly containing one or more hetero atoms chosen from N, O, S and P; and

A⁻ is a physiologically acceptable counterion.

As regards the meaning of Y, the term “hydrophilic group” means a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulphonated polyesters, sulphonated polyamides or a mixture of these polymers. The hydrophilic compound is preferentially a polyether and in particular a poly(ethylene oxide) or poly(propylene oxide).

The cationic associative polyurethanes of formula (XVIII) which can be used according to the invention are formed from diisocyanates and from various compounds with functions containing a labile hydrogen. The functions containing a labile hydrogen may be alcohol, primary or secondary amine or thiol functions, giving, after reaction with the diisocyanate functions, polyurethanes, polyureas and polythioureas, respectively. The expression “polyurethanes which can be used according to the present invention” encompasses these three types of polymer, namely polyurethanes per se, polyureas and polythioureas, and also copolymers thereof.

A first type of compound involved in the preparation of the polyurethane of formula (XVIII) is a compound comprising at least one unit containing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:

HZ—(P)ₙ—ZH

or

HZ—(P′)ₚ—ZH

in which Z, P, P′, n and p are as defined above.

Examples of compounds containing an amine function that may be mentioned include N-methyldiethanolamine, N-tert-butyldiethanolamine and N-sulphoethyldiethanolamine.

The second compound involved in the preparation of the polyurethane of formula (XVIII) is a diisocyanate corresponding to the formula:

O═C═N—R₄—N═C═O

in which R₄ is as defined above.

By way of example, mention may be made of methylenediphenyl diisocyanate, methylenecyclohexane diisocyanate, isophorone diisocyanate, toluene diisocyanate, naphthalene diisocyanate, butane diisocyanate and hexane diisocyanate.

A third compound involved in the preparation of the polyurethane of formula (XVIII) is a hydrophobic compound intended to form the terminal hydrophobic groups of the polymer of formula (XVIII).

This compound consists of a hydrophobic group and of a function containing a labile hydrogen, for example a hydroxyl, primary or secondary amine, or thiol function.

By way of example, this compound may be a fatty alcohol such as, in particular, stearyl alcohol, dodecyl alcohol or decyl alcohol. When this compound comprises a polymeric chain, it may be, for example, α-hydroxylated hydrogenated polybutadiene.

The hydrophobic group of the polyurethane of formula (XVIII) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulphate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, for example, of polyethers, sulphonated polyesters and sulphonated polyamides, or a mixture of these polymers. The hydrophilic compound is preferentially a polyether and especially a poly(ethylene oxide) or poly(propylene oxide).

The hydrophilic group termed Y in formula (XVIII) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.

(II) quaternized cellulose derivatives and polyacrylates containing non-cyclic amine side groups.

The quaternized cellulose derivatives are, in particular:

quaternized celluloses modified with groups comprising at least one fatty chain, such as alkyl, arylalkyl or alkylaryl groups containing at least 8 carbon atoms, or mixtures thereof.

quaternized hydroxyethylcelluloses modified with groups comprising at least one fatty chain, such as alkyl, arylalkyl or alkylaryl groups containing at least 8 carbon atoms, or mixtures thereof.

The alkyl radicals borne by the above quaternized celluloses or hydroxyethylcelluloses preferably contain from 8 to 30 carbon atoms. The aryl radicals preferably denote phenyl, benzyl, naphthyl or anthryl groups.

Examples of quaternized alkylhydroxyethylcelluloses containing C₈-C₃₀ fatty chains that may be mentioned include the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Amerchol, and the products Crodacel QM®, Crodacel QL® (C₁₂ alkyl) and Crodacel QS® (C₁₈ alkyl) sold by the company Croda.

### Amphoteric Associative Polymers

The amphoteric associative polymers are preferably chosen from those comprising at least one non-cyclic cationic unit. Even more particularly, the ones that are preferred are those prepared from or comprising 1 to 20 mol %, preferably 1.5 to 15 mol % and even more particularly 1.5 to 6 mol % of fatty-chain monomer relative to the total number of moles of monomers.

The amphoteric associative polymers that are preferred according to the invention comprise or are prepared by copolymerizing:

1) at least one monomer of formula (XIX) or (XX):

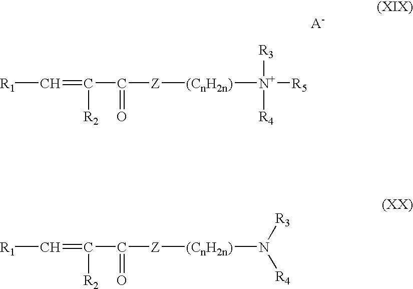

in which R₁ and R₂, which may be identical or different, represent a hydrogen atom or a methyl radical, R₃, R₄ and R₅, which may be identical or different, represent a linear or branched alkyl radical containing from 1 to 30 carbon atoms,

Z represents an NH group or an oxygen atom,

n is an integer from 2 to 5,

A⁻ is an anion derived from an organic or mineral acid, such as a methosulphate anion or a halide such as chloride or bromide;

2) at least one monomer of formula (XXI)

R₆—CH═CR₇—COOH (XXI)

in which R₆ and R₇, which may be identical or different, represent a hydrogen atom or a methyl radical;

and

3) at least one monomer of formula (XXII):

R₆—CH═CR₇—COXR₈ (XXII)

in which R₆ and R₇, which may be identical or different, represent a hydrogen atom or a methyl radical, X denotes an oxygen or nitrogen atom and R₈ denotes a linear or branched alkyl radical containing from 1 to 30 carbon atoms;

at least one of the monomers of formula (XIX), (XX) or (XXII) comprising at least one fatty chain.

The monomers of formulae (XIX) and (XX) of the present invention are preferably chosen from the group consisting of:

dimethylaminoethyl methacrylate, dimethylaminoethyl acrylate,

diethylaminoethyl methacrylate, diethylaminoethyl acrylate,

dimethylaminopropyl methacrylate, dimethylaminopropyl acrylate,

dimethylaminopropylmethacrylamide, dimethylaminopropylacrylamide,

these monomers optionally being quaternized, for example with a C₁-C₄ alkyl halide or a C₁-C₄ dialkyl sulphate.

More particularly, the monomer of formula (XIX) is chosen from acrylamidopropyltrimethylammonium chloride and methacrylamidopropyltrimethylammonium chloride.

The monomers of formula (XXI) of the present invention are preferably chosen from the group consisting of acrylic acid, methacrylic acid, crotonic acid and 2-methylcrotonic acid. More particularly, the monomer of formula (XXI) is acrylic acid.

The monomers of formula (XXII) of the present invention are preferably chosen from the group consisting of C₁₂-C₂₂ and more particularly C₁₆-C₁₈ alkyl acrylates or methacrylates.

The monomers constituting the fatty-chain amphoteric polymers of the invention are preferably already neutralized and/or quaternized.

The ratio of the number of cationic charges/anionic charges is preferably equal to about 1.

The amphoteric associative polymers according to the invention preferably comprise from 1 mol % to 10 mol % of the monomer comprising a fatty chain (monomer of formula (XIX), (XX) or (XXII)), and preferably from 1.5 mol % to 6 mol %.

The weight-average molecular weights of the amphoteric associative polymers according to the invention may range from 500 to 50 000 000 and are preferably between 10 000 and 5 000 000.

The amphoteric associative polymers according to the invention may also contain other monomers such as nonionic monomers and in particular such as C₁-C₄ alkyl acrylates or methacrylates.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type used according to the invention are preferably chosen from:

(1) celluloses modified with groups comprising at least one fatty chain;

examples that may be mentioned include:

hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product Natrosol Plus Grade 330 CS® (C₁₆ alkyls) sold by the company Aqualon, or the product Bermocoll EHM 100® sold by the company Berol Nobel,

those modified with alkylphenyl polyalkylene glycol ether groups, such as the product Amercell Polymer HM-1500® (nonylphenyl polyethylene glycol (15) ether) sold by the company Amerchol.

(2) hydroxypropyl guars modified with groups comprising at least one fatty chain, such as the product Esaflor HM 22® (C₂₂ alkyl chain) sold by the company Lamberti, and the products RE210-18® (C₁₄ alkyl chain) and RE205-1® (C₂₀ alkyl chain) sold by the company Rhône-Poulenc.

(3) copolymers of vinylpyrrolidone and of fatty-chain hydrophobic monomers; examples that may be mentioned include:

the products Antaron V216® or Ganex V216® (vinylpyrrolidone/hexadecene copolymer) sold by the company I.S.P.

the products Antaron V220® or Ganex V220® (vinylpyrrolidone/eicosene copolymer) sold by the company I.S.P.

(4) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®.

(5) copolymers of hydrophilic methacrylates or acrylates and of hydrophobic monomers comprising at least one fatty chain, such as, for example, the polyethylene glycol methacrylate/lauryl methacrylate copolymer.

(6) polyurethane polyethers comprising in their chain both hydrophilic blocks usually of polyoxyethylenated nature and hydrophobic blocks which may be aliphatic sequences alone and/or cycloaliphatic and/or aromatic sequences.

(7) polymers with an aminoplast ether skeleton containing at least one fatty chain, such as the Pure Thix® compounds sold by the company Sud-Chemie.

Preferably, the polyurethane polyethers comprise at least two hydrocarbon-based lipophilic chains containing from 6 to 30 carbon atoms, separated by a hydrophilic block, the hydrocarbon-based chains possibly being pendent chains, or chains at the end of the hydrophilic block. In particular, it is possible for one or more pendent chains to be included. In addition, the polymer may comprise a hydrocarbon-based chain at one end or at both ends of a hydrophilic block.

The polyurethane polyethers may be multiblock, in particular in triblock form. Hydrophobic blocks may be at each end of the chain (for example: triblock copolymer with a hydrophilic central block) or distributed both at the ends and in the chain (for example: multiblock copolymer). These same polymers may also be graft polymers or starburst polymers.

The nonionic fatty-chain polyurethane polyethers may be triblock copolymers in which the hydrophilic block is a polyoxyethylenated chain comprising from 50 to 1 000 oxyethylene groups. The nonionic polyurethane polyethers comprise a urethane linkage between the hydrophilic blocks, whence arises the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂₋₁₄ alkyl chain, and the product Elfacos T212® containing a C₁₈ alkyl chain, from Akzo.

The product DW 1206B® from Rohm & Haas containing a C₂₀ alkyl chain and a urethane linkage, sold at a solids content of 20% in water, may also be used.

It is also possible to use solutions or dispersions of these polymers, especially in water or in aqueous-alcoholic medium. Examples of such polymers that may be mentioned are Rheolate® 255, Rheolate® 278 and Rheolate® 244 sold by the company Rheox. The products DW 1206F and DW 1206J sold by the company Rohm & Haas may also be used.

The polyurethane polyethers that may be used according to the invention are in particular those described in the article by G. Fonnum, J. Bakke and Fk. Hansen—Colloid Polym. Sci 271, 380.389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold especially by the company Rohm & Haas under the names Aculyn 44® and Aculyn 46® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexylisocyanate) (SMDI), at 35% by weight in a mixture of propylene glycol (39%) and water (26%)].

According to a third preferred mode, the composition according to the present invention also contains at least one additional surfactant.

The surfactants that are suitable for carrying out the present invention are especially the following:

(i) Anionic Surfactant(s):

By way of example of anionic surfactants that can be used, alone or as mixtures, in the context of the present invention, mention may be made in particular (non-limiting list) of salts (in particular alkali metal salts, especially sodium salts, ammonium salts, amine salts, amino alcohol salts or magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; (C₆-C₂₄)alkyl sulphosuccinates, (C₆-C₂₄)alkyl ether sulphosuccinates, (C₆-C₂₄)alkylamide sulphosuccinates; (C₆-C₂₄)alkyl sulphoacetates; (C₆-C₂₄)acyl sarcosinates; and (C₆-C₂₄)acyl glutamates. It is also possible to use (C₆-C₂₄)alkylpolyglycoside carboxylic esters such as alkylglucoside citrates, alkylpolyglycoside tartrates and alkylpolyglycoside sulphosuccinates, alkylsulphosuccinamates; acyl isethionates and N-acyl taurates, the alkyl or acyl radical of all of these different compounds preferably containing from 12 to 20 carbon atoms and the aryl radical preferably denoting a phenyl or benzyl group. Among the anionic surfactants which can also be used, mention may also be made of fatty acid salts such as oleic, ricinoleic, palmitic and stearic acid salts, coconut oil acid or hydrogenated coconut oil acid; acyl lactylates in which the acyl radical contains 8 to 20 carbon atoms. It is also possible to use alkyl D-galactoside uronic acids and their salts, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, in particular those containing from 2 to 50 alkylene oxide groups, in particular ethylene oxide groups, and mixtures thereof.

(ii) Additional Nonionic Surfactant(s):

These additional nonionic surfactants are different from the polyol esters used as essential components in the compositions according to the invention.

The additional nonionic surfactants are, themselves also, compounds that are well known per se (see in particular in this respect “Handbook of Surfactants” by M. R. Porter, published by Blackie & Son (Glasgow and London), 1991, pp. 116-178) and their nature is not a critical factor in the context of the present invention. Thus, they can be chosen in particular from (non-limiting list) polyethoxylated or polypropoxylated, alkylphenols, alpha-diols or alcohols, having a fatty chain containing, for example, 8 to 18 carbon atoms, it being possible for the number of ethylene oxide or propylene oxide groups to range in particular from 2 to 50. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; alkylpolyglycosides, N-alkylglucamine derivatives, and amine oxides such as (C₁₀-C₁₄)alkylamine oxides or N-acylaminopropylmorpholine oxides.

(iii) Amphoteric or Zwitterionic Surfactant(s):

The amphoteric or zwitterionic surfactants, the nature of which is not a critical factor in the context of the present invention, can be, in particular (non-limiting list), aliphatic secondary or tertiary amine derivatives in which the aliphatic radical is a linear or branched chain containing 8 to 18 carbon atoms and containing at least one water-solubilizing anionic group (for example carboxylate, sulphonate, sulphate, phosphate or phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives, mention may be made of the products sold under the name Miranol, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures:

R₂—CONHCH₂CH₂—N(R₃)(R₄)(CH₂COO⁻)

in which: R₂ denotes an alkyl radical of an acid R₂—COOH present in hydrolysed coconut oil, a heptyl, nonyl or undecyl radical, R₃ denotes a beta-hydroxyethyl group and R₄ denotes a carboxymethyl group;

and

R₂′—CONHCH₂CH₂—N(B)(C)

in which:

B represents —CH₂CH₂OX′, C represents —(CH₂)_{z}—Y′, with z=1 or 2,

X′ denotes the —CH₂CH₂—COOH group or a hydrogen atom,

Y′ denotes —COOH or the —CH₂—CHOH—SO₃H radical,

R₂′ denotes an alkyl radical of an acid R₉—COOH present in coconut oil or in hydrolysed linseed oil, an alkyl radical, in particular a C₇, C₉, C₁₁ or C₁₃ alkyl radical, a C₁₇ alkyl radical and its iso form, or an unsaturated C₁₇ radical.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

(iv) Cationic Surfactants:

Among the cationic surfactants, mention may be made in particular (non-limiting list) of: salts of optionally polyoxyalkylenated primary, secondary or tertiary fatty amines; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

The amounts of additional surfactants (other than polyol esters) present in the composition according to the invention can range from 0.01% to 40% and preferably from 0.5% to 30% relative to the total weight of the composition.

The composition of the present invention may also comprise one or more additional oxidation bases conventionally used in oxidation dyeing other than the para-phenylenediamines of formula I. By way of example, these additional oxidation bases are chosen from para-phenylenediamines, bis(phenyl)alkylenediamines, para-aminophenols, ortho-aminophenols and heterocyclic bases other than the heterocyclic para-phenylenediamines which are useful for the invention, and the addition salts thereof.

Among the para-phenylenediamines that may be mentioned, for example, are para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis-(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine, 2,3-diamino-6-methoxypyridine, 2-(β-methoxyethyl)amino-3-amino-6-methoxy pyridine, and 3,4-diaminopyridine, and the addition salts thereof.

Other pyridine oxidation bases that are useful in the present invention are the 3-aminopyrazolo[1,5-a]pyridine oxidation bases or addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamino, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol,

and the addition salts thereof.

Among the pyrimidine derivatives that may be mentioned are the compounds described, for example, in patents DE 2 359 399; JP 88-169 571; JP 05-63124; EP 0 770 375 or patent application WO 96/15765, for instance 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and the addition salts thereof, and the tautomeric forms thereof, when a tautomeric equilibrium exists.

Among the pyrazole derivatives that may be mentioned are the compounds described in patents DE 3 843 892 and DE 4 133 957, and patent applications WO 94/08969, WO 94/08970, FR-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof.

The additional oxidation base(s) present in the composition of the invention is (are) generally present in an amount ranging from 0.001% to 20% by weight approximately, and preferably ranging from 0.005% to 6%, relative to the total weight of the dye composition.

The composition according to the invention preferably contains one or more additional couplers conventionally used for dyeing keratin fibres. Among these couplers, mention may be made especially of meta-phenylenediamines, meta-diphenols, naphthalene-based couplers and heterocyclic couplers, and the addition salts thereof.

Examples that may be mentioned include 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

In the composition of the present invention, the coupler(s) is (are) generally present in an amount ranging from 0.001% to 20% and preferably ranging from 0.005% to 6% by weight approximately relative to the total weight of the dye composition.

In general, the addition salts of the oxidation bases and couplers that may be used in the context of the invention are chosen especially from the addition salts with an acid, such as the hydrochlorides, hydrobromides, sulphates, citrates, succinates, tartrates, lactates, tosylates, benzenesulphonates, phosphates and acetates, and the addition salts with a base, such as sodium hydroxide, potassium hydroxide, ammonia, amines or alkanolamines.

The dye composition in accordance with the invention may also contain one or more direct dyes, which may especially be chosen from neutral, acidic or cationic nitrobenzene direct dyes, neutral, acidic or cationic azo direct dyes, neutral, acidic or cationic quinone and in particular anthraquinone direct dyes, azine direct dyes, triarylmethane direct dyes, indoamine direct dyes and natural direct dyes.

Among the benzenic direct dyes that may be used according to the invention, mention may be made, in a non-limiting manner, of the following compounds:

1,4-diamino-2-nitrobenzene

1-amino-2-nitro-4-(β-hydroxyethylamino)benzene

1-amino-2-nitro-4-bis(β-hydroxyethyl)aminobenzene

1,4-bis(β-hydroxyethylamino)-2-nitrobenzene

1-β-hydroxyethylamino-2-nitro-4-bis(β-hydroxyethylamino)benzene

1-β-hydroxyethylamino-2-nitro-4-aminobenzene

1-β-hydroxyethylamino-2-nitro-4-(ethyl)(β-hydroxyethyl)aminobenzene

1-amino-3-methyl-4-β-hydroxyethylamino-6-nitrobenzene

1-amino-2-nitro-4-β-hydroxyethylamino-5-chlorobenzene

1,2-diamino-4-nitrobenzene

1-amino-2-β-hydroxyethylamino-5-nitrobenzene

1,2-bis(β-hydroxyethylamino)-4-nitrobenzene

1-amino-2-[tris(hydroxymethyl)methylamino]-5-nitrobenzene

1-hydroxy-2-amino-5-nitrobenzene

1-hydroxy-2-amino-4-nitrobenzene

1-hydroxy-3-nitro-4-aminobenzene

1-hydroxy-2-amino-4,6-dinitrobenzene

1-β-hydroxyethyloxy-2-β-hydroxyethylamino-5-nitrobenzene

1-methoxy-2-β-hydroxyethylamino-5-nitrobenzene

1-β-hydroxyethyloxy-3-methylamino-4-nitrobenzene

1-β,γ-dihydroxypropyloxy-3-methylamino-4-nitrobenzene

1-β-hydroxyethylamino-4-β,γ-dihydroxypropyloxy-2-nitrobenzene

1-β,γ-dihydroxypropylamino-4-trifluoromethyl-2-nitrobenzene

1-β-hydroxyethylamino-4-trifluoromethyl-2-nitrobenzene

1-β-hydroxyethylamino-3-methyl-2-nitrobenzene

1-β-aminoethylamino-5-methoxy-2-nitrobenzene

1-hydroxy-2-chloro-6-ethylamino-4-nitrobenzene

1-hydroxy-2-chloro-6-amino-4-nitrobenzene

1-hydroxy-6-[bis(β-hydroxyethyl)amino]-3-nitrobenzene

1-β-hydroxyethylamino-2-nitrobenzene

1-hydroxy-4-β-hydroxyethylamino-3-nitrobenzene.

Among the azo direct dyes that may be used according to the invention, mention may be made of the cationic azo dyes described in patent applications WO 95/15144, WO 95/01772 and EP 714 954, the content of which forms an integral part of the invention.

Among these compounds, mention may be made most particularly of the following dyes:

1,3-dimethyl-2-[[4-(dimethylamino)phenyl]azo]-1H-imidazolium chloride,

1,3-dimethyl-2-[(4-aminophenyl)azo]-1H-imidazolium chloride,

1-methyl-4-[(methylphenylhydrazono)methyl]pyridinium methyl sulphate.

Among the azo direct dyes that may also be mentioned are the following dyes described in the Colour Index International 3rd edition:

Disperse Red 17

Acid Yellow 9

Acid Black 1

Basic Red 22

Basic Red 76

Basic Yellow 57

Basic Brown 16

Acid Yellow 36

Acid Orange 7

Acid Red 33

Acid Red 35

Basic Brown 17

Acid Yellow 23

Acid Orange 24

Disperse Black 9.

Mention may also be made of 1-(4′-aminodiphenylazo)-2-methyl-4-[bis(β-hydroxyethyl)amino]benzene and 4-hydroxy-3-(2-methoxy-phenylazo)-1-naphthalenesulphonic acid.

Among the quinone direct dyes that may be mentioned are the following dyes:

Disperse Red 15

Solvent Violet 13

Acid Violet 43

Disperse Violet 1

Disperse Violet 4

Disperse Blue 1

Disperse Violet 8

Disperse Blue 3

Disperse Red 11

Acid Blue 62

Disperse Blue 7

Basic Blue 22

Disperse Violet 15

Basic Blue 99

and also the following compounds:

1-N-methylmorpholiniumpropylamino-4-hydroxyanthraquinone

1-aminopropylamino-4-methylaminoanthraquinone

1-aminopropylaminoanthraquinone

5-β-hydroxyethyl-1,4-diaminoanthraquinone

2-aminoethylaminoanthraquinone

1,4-bis(β,γ-dihydroxypropylamino)anthraquinone.

Among the azine dyes that may be mentioned are the following compounds:

Basic Blue 17

Basic Red 2.

Among the triarylmethane dyes that may be used according to the invention, mention may be made of the following compounds:

Basic Green 1

Acid Blue 9

Basic Violet 3

Basic Violet 14

Basic Blue 7

Acid Violet 49

Basic Blue 26

Acid Blue 7.

Among the indoamine dyes that may be used according to the invention, mention may be made of the following compounds:

2-β-hydroxyethylamino-5-[bis(β-4′-hydroxyethyl)amino]anilino-1,4-benzoquinone;

2-β-hydroxyethylamino-5-(2′-metnoxy-4′-amino)anilino-1,4-benzoquinone;

3-N(2′-chloro-4′-hydroxy)phenylacetylamino-6-methoxy-1,4-benzoquinoneimine;

3-N(3′-chloro-4′-methylamino)phenylureido-6-methyl-1,4-benzoquinoneimine;

3-[4′-N-(ethylcarbamylmethyl)amino]phenylureido-6-methyl-1,4-benzoquinoneimine.

Among the natural direct dyes that may be used according to the invention, mention may be made of lawsone, juglone, alizarin, purpurin, carminic acid, kermesic acid, purpurogallin, protocatechaldehyde, indigo, isatin, curcumin, spinulosin and apigenidin. Extracts or decoctions containing these natural dyes may also be used, and especially henna-based poultices or extracts.

The direct dye(s) preferably represent(s) from 0.001% to 20% by weight approximately, and even more preferably from 0.005% to 10% by weight approximately, relative to the total weight of the ready-to-use composition.

The composition according to the invention may also contain at least one hydroxylated solvent, for instance ethanol, propylene glycol, glycerol, polyol monoethers or benzyl alcohol.

It may also contain a non-hydroxylated solvent.

The hydroxylated solvents and non-hydroxylated solvents are preferably present in proportions preferably of between 1% and 40% by weight approximately and even more preferably between 5% and 30% by weight approximately relative to the total weight of the dye composition.

The dye composition in accordance with the invention may also contain various adjuvants conventionally used in hair dye compositions, such as antioxidants, penetrating agents, sequestering agents, fragrances, buffers, dispersants, conditioners, for instance volatile or non-volatile, modified or unmodified silicones, film-forming agents, ceramides, preserving agents and opacifiers.

The above adjuvants are generally present in an amount for each of between 0.01% and 20% by weight relative to the weight of the composition.

Needless to say, a person skilled in the art will take care to select this or these optional additional compound(s) such that the advantageous properties intrinsically associated with the oxidation dye composition in accordance with the invention are not, or are not substantially, adversely affected by the envisaged addition(s).

The pH of the dye composition in accordance with the invention is generally between 3 and 12 approximately and preferably between 5 and 11 approximately. It may be adjusted to the desired value by means of acidifying or basifying agents usually used for dyeing keratin fibres, or alternatively using standard buffer systems.

Among the acidifying agents that may be mentioned, for example, are mineral or organic acids, for instance hydrochloric acid, orthophosphoric acid, sulphuric acid, carboxylic acids, for instance acetic acid, tartaric acid, citric acid or lactic acid, and sulphonic acids.

Among the basifying agents that may be mentioned, for example, are aqueous ammonia, alkaline carbonates, alkanolamines such as monoethanolamine, diethanolamine and triethanolamine and derivatives thereof, sodium hydroxide, potassium hydroxide and the compounds of formula (XXIII) below:

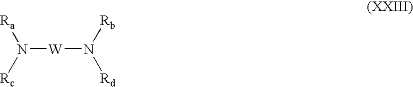

in which W is a propylene residue optionally substituted with a hydroxyl group or a C₁-C₄ alkyl radical; Rₐ, R_{b}, R_{c}, and R_{d}, which may be identical or different, represent a hydrogen atom or a C₁-C₄ alkyl or C₁-C₄ hydroxyalkyl radical.

The dye composition according to the invention may be in various forms, such as in the form of liquids, creams or gels, or in any other form that is suitable for dyeing keratin fibres, and especially human hair.

The process of the present invention is a process in which the composition according to the present invention as defined above is applied to the fibres, and the colour is developed using an oxidizing agent. The colour may be revealed at acidic, neutral or alkaline pH and the oxidizing agent may be added to the composition of the invention just at the time of use, or it may be introduced using an oxidizing composition containing it, applied simultaneously with or sequentially to the composition of the invention.

According to one particular embodiment, the composition according to the present invention is mixed, preferably at the time of use, with a composition containing, in a medium that is suitable for dyeing, at least one oxidizing agent, this oxidizing agent being present in an amount that is sufficient to develop a coloration. The mixture obtained is then applied to the keratin fibres. After a leave-in time of 3 to 50 minutes approximately and preferably 5 to 30 minutes approximately, the keratin fibres are rinsed, washed with shampoo, rinsed again and then dried.

The oxidizing agents conventionally used for the oxidation dyeing of keratin fibres are, for example, hydrogen peroxide, urea peroxide, alkali metal bromates, persalts such as perborates and persulphates, peracids, and oxidase enzymes, among which mention may be made of peroxidases, 2-electron oxidoreductases such as uricases, and 4-electron oxygenases, for instance laccases. Hydrogen peroxide is particularly preferred.

The oxidizing composition may also contain various adjuvants conventionally used in hair dye compositions and as defined above.

The pH of the oxidizing composition containing the oxidizing agent is such that, after mixing with the dye composition, the pH of the resulting composition applied to the keratin fibres preferably ranges between 3 and 12 approximately and even more preferably between 5 and 11. It may be adjusted to the desired value by means of acidifying or basifying agents usually used in the dyeing of keratin fibres and as defined above.

The ready-to-use composition that is finally applied to the keratin fibres may be in various forms, such as in the form of liquids, creams or gels, or in any other form that is suitable for dyeing keratin fibres, and especially human hair.

A subject of the invention is also a multi-compartment device or dyeing “kit”, in which a first compartment contains the dye composition defined above and a second compartment contains an oxidizing agent. This device may be equipped with a means for applying the desired mixture to the hair, such as the devices described in patent FR 2 586 913 in the name of the Applicant.

Using this device, it is possible to dye keratin fibres using a process that involves mixing a dye composition of the invention with an oxidizing agent as defined above, and applying the mixture obtained to the keratin fibres for a time that is sufficient to develop the desired coloration.

### DETAILED DESCRIPTION

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Dye Composition: (Expressed in Grams)

| | |
|---|---|
| Oleyl alcohol | 6 |
| Oleic acid | 3 |
| Oleyl alcohol polyglycerolated with 2 mol of glycerol | 6 |
| Oleyl alcohol polyglycerolated with 6 mol of glycerol | 6 |
| Diethylaminopropyl laurylaminosuccinamate, sodium salt | 3 |
| Oleylamine oxyethylenated with 2 mol of ethylene oxide | 7 |
| Carboxylic acid alkyl ether monoethanolamide containing | 10 |
| 2 mol of ethylene oxide | |
| Polysorbate 20 | 2.5 |
| Ammonium acetate | 20 |
| Propylene glycol | 20 |
| Dilinoleic acid | 1.5 |
| Reducing agents, antioxidants | 0.915 |
| Sequestering agents | 1 |
| 1,3-Dihydroxybenzene (resorcinol) | 0.085 |
| [1-(4-Aminophenyl)pyrrolidin-3-yl]trimethylammonium chloride | 1.0 |
| 2-Methyl-5-aminophenol | 0.5 |
| Niacinamide | 0.2 |
| Fragrance | Qs |
| Aqueous ammonia (containing 20.5% ammonia) | 10.2 |
| Demineralized water qs | 100 |

This composition was mixed weight for weight, at the time of use, with an oxidizing milk containing 6% hydrogen peroxide. The mixture obtained was applied for 30 minutes to grey hair containing 90% white hairs. After rinsing, shampooing and drying, a violet-brown coloration was obtained on this hair.

### EXAMPLE 2

Dye Composition: (Expressed in Grams)

| | |
|---|---|
| Oleyl alcohol | 4 |
| Oleic acid | 5 |
| Oleyl alcohol polyglycerolated with 2 mol of glycerol | 4 |
| Lauryl alcohol polyglycerolated with 4 mol of glycerol | 3.6 |
| Rapeseed acid amide oxyethylenated with 4 mol of ethylene | 8 |
| oxide | |
| Oleylamine oxyethylenated with 2 mol of ethylene oxide | 4 |
| Decyl alcohol oxyethylenated with 3 mol of ethylene oxide | 2.7 |
| PEG-4 oleate | 2.5 |
| Propylene glycol | 20 |
| Adipic acid | 1.3 |
| Reducing agents, antioxidants | 0.63 |
| Sequestering agent | 1 |
| 3-[1-(4-Aminophenyl)pyrrolidin-3-yl]-1-methyl-3H- | 0.8 |
| imidazol-1-ium chloride | |
| 5N-(β-Hydroxyethyl)amino-2-methylphenol | 0.4 |
| Pure monoethanolamine | 2 |
| Panthenol | 0.1 |
| Fragrance | qs |
| Aqueous ammonia (containing 20.5% ammonia) | 10 |
| Demineralized water qs | 100 |

This composition was mixed weight for weight, at the time of use, with an oxidizing milk containing 6% hydrogen peroxide. The mixture obtained was applied for 30 minutes to grey hair containing 90% white hairs. After rinsing, shampooing and drying, a violet coloration was obtained on this hair.

What is claimed is:

1. A dye composition for dyeing keratin fibres, comprising, in a medium that is suitable for dyeing, at least one cationic tertiary para-phenylenediamine comprising a pyrrolidine nucleus and at least one polyol ester.

2. The composition of claim 1, wherein the cationic tertiary para-phenylenediamine corresponds to formula (I): 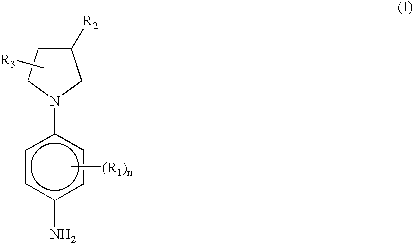
in which
n ranges from 0 to 4, it being understood that when n is greater than or equal to 2, then the radicals R₁ may be identical or different,
R₁ represents a halogen atom; a C₁-C₆ aliphatic or alicyclic, saturated or unsaturated hydrocarbon-based chain, the chain possibly being interrupted with one or more oxygen, nitrogen, silicon or sulphur atoms or by an SO₂ group, and possibly being substituted with one or more hydroxyl or amino radicals; an onium radical Z, the radical R₁ not comprising a peroxide bond or diazo, nitro or nitroso radicals,
R₂ represents an onium radical Z or a radical —X—C═NR₈—NR₉R₁₀ in which X represents an oxygen atom or a radical —NR₁₁ and R₈, R₉, R₁₀ and R₁₁ represent a hydrogen atom, a C₁-C₄ alkyl radical or a C₁-C₄ hydroxyalkyl radical,
R₃ represents a hydrogen atom or a hydroxyl radical.

3. The composition of claim 2, wherein the cationic tertiary para-phenylenediamine is such that n is equal to 0.

4. The composition of claim 2, wherein the cationic tertiary para-phenylenediamine is such that n is equal to 1 and R₁ is chosen from the group formed by a halogen atom; a C₁-C₆ aliphatic or alicyclic, saturated or unsaturated hydrocarbon-based chain; one or more carbon atoms possibly being replaced with an oxygen, nitrogen, silicon or sulphur atom or with an SO₂ group, the radical R₁ not comprising a peroxide bond or diazo, nitro or nitroso radicals.

5. The composition of claim 2, wherein the cationic tertiary para-phenylenediamine is such that R₁ is chosen from chlorine, bromine and C₁-C₄ alkyl, C₁-C₄ hydroxyalkyl, C₁-C₄ aminoalkyl, C₁-C₄ alkoxy or C₁-C₄ hydroxyalkoxy radicals.

6. The composition of claim 5, wherein the cationic tertiary para-phenylenediamine is such that R₁ is chosen from a methyl, hydroxymethyl, 2-hydroxyethyl, 1,2-dihydroxyethyl, methoxy, isopropyloxy or 2-hydroxyethoxy radical.

7. The composition of claim 2, wherein the cationic tertiary para-phenylenediamine is such that R₂ represents the onium radical Z corresponding to formula (II) 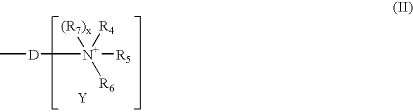
in which
D is a single bond or a linear or branched C₁-C₁₄ alkylene chain which may be interrupted with one or more hetero atoms chosen from oxygen, sulphur and nitrogen, and which may be substituted with one or more hydroxyl, C₁-C₆ alkoxy or amino radicals, and which may bear one or more ketone functions;
R₄, R₅ and R₆, taken separately, represent a C₁-C₁₅ alkyl radical; a C₁-C₆ monohydroxyalkyl radical; a C₂-C₆ polyhydroxyalkyl radical; a (C₁-C₆)alkoxy(C₁-C₆)alkyl radical; an aryl radical; a benzyl radical; a C₁-C₆ amidoalkyl radical; a tri(C₁-C₆)alkylsilane(C₁-C₆)alkyl radical; a C₁-C₆ aminoalkyl radical; a C₁-C₆ aminoalkyl radical in which the amine is mono- or disubstituted with a C₁-C₄ alkyl, (C₁-C₆)alkylcarbonyl, amido or (C₁-C₆)alkylsulphonyl radical; or
R₄, R₅ and R₆ together, in pairs, form, with the nitrogen atom to which they are attached, a saturated 4-, 5-, 6- or 7-membered carbon-based ring optionally containing one or more hetero atoms, the cationic ring possibly being substituted with a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a (C₁-C₆)alkylcarbonyl radical, a thio (—SH) radical, a C₁-C₆ thioalkyl (—R—SH) radical, a (C₁-C₆)alkylthio radical, an amino radical, an amino radical mono- or disubstituted with a (C₁-C₆)alkyl, (C₁-C₆)alkylcarbonyl, amido or (C₁-C₆)alkylsulphonyl radical;
R₇ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;
x is 0 or 1,
when x=0, then the linker arm is attached to the nitrogen atom bearing the radicals R₄ to R₆,
when x=1, then two of the radicals R₄ to R₆ form, together with the nitrogen atom to which they are attached, a 4-, 5-, 6- or 7-membered saturated ring and D is linked to a carbon atom of the saturated ring;
Y is a counterion.

8. The composition of claim 7, wherein the cationic tertiary para-phenylenediamine is such that R₂ corresponds to formula II in which x is equal to 0 and R₄, R₅ and R₆, separately, are preferably chosen from a C₁-C₆ alkyl radical, a C₁-C₄ monohydroxyalkyl radical, a C₂-C₄ polyhydroxyalkyl radical, a (C₁-C₆)alkoxy(C₁-C₄)alkyl radical, a C₁-C₆ amidoalkyl radical, a tri(C₁-C₆)alkylsilane(C₁-C₆)alkyl radical, or R₄ and R₅ together form an azetidine, pyrrolidine, piperidine, piperazine or morpholine ring, R₆ being chosen in this case from a C₁-C₆ alkyl radical; a C₁-C₆ monohydroxyalkyl radical; a C₂-C₆ polyhydroxyalkyl radical; a C₁-C₆ aminoalkyl radical, an aminoalkyl radical mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

9. The composition of claim 7, wherein the cationic tertiary para-phenylenediamine is such that R₂ corresponds to formula II in which x is equal to 1 and R₇ is chosen from a C₁-C₆ alkyl radical; a C₁-C₆ monohydroxyalkyl radical; a C₂-C₆ polyhydroxyalkyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₄ and R₅ together form an azetidine, pyrrolidine, piperidine, piperazine or morpholine ring, R₆ being chosen in this case from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarboxy(C₁-C₆)alkyl radical; a (C₁-C₆)alkylcarbonyl(C₁-C₆)alkyl radical; an N—(C₁-C₆)alkylcarbamyl(C₁-C₆)alkyl radical.

10. The composition of claim 2, wherein the cationic tertiary para-phenylenediamine is such that D is a single bond or an alkylene chain that may be substituted.

11. The composition of claim 7, wherein the cationic tertiary para-phenylenediamine is such that R₂ is a trialkylammonium radical.

12. The composition of claim 2, wherein the cationic tertiary para-phenylenediamine is such that R₂ represents the onium radical Z corresponding to formula (III) 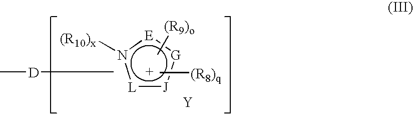
in which
D is a single bond or a linear or branched C₁-C₁₄ alkylene chain that may be interrupted with one or more hetero atoms chosen from oxygen, sulphur and nitrogen, and that may be substituted with one or more hydroxyl, C₁-C₆ alkoxy or amino radicals, and that may bear one or more ketone functions;
the fing members E, G, J and L, which may be identical or different, represent a carbon, oxygen, sulphur or nitrogen atom to form a pyrrole, pyrazole, imidazole, triazole, oxazole, isoxazole, thiazole or isothiazole ring,
q is an integer between 0 and 4 inclusive;
o is an integer between 0 and 3 inclusive;
q+o is an integer between 0 and 4;
the radicals R₈, which may be identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical mono- or disubstituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₈ are borne by a carbon atom,
the radicals R₉, which may be identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical or a benzyl radical; it being understood that the radicals R₉ are borne by a nitrogen atom,
R₁₀ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical, a C₁-C₆ aminoalkyl radical in which the amine is substituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;
x is 0 or 1
when x=0, the linker arm D is attached to the nitrogen atom,
when x=1, the linker arm D is attached to one of the ring members E, G, J or L,
Y is a counterion.

13. The composition of claim 12, wherein the cationic tertiary para-phenylenediamine is such that the ring members E, G, J and L form an imidazole ring.

14. The composition of claim 12, wherein the cationic tertiary para-phenylenediamine is such that x is equal to 0 and D is a single bond or an alkylene chain that may be substituted.

15. The composition of claim 2, wherein the cationic tertiary para-phenylenediamine is such that R₂ represents an onium radical Z corresponding to formula (IV) 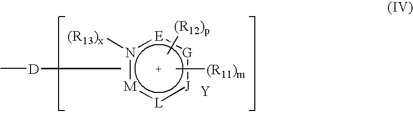
in which:
D is a single bond or a linear or branched C₁-C₁₄ alkylene chain which may be interrupted with one or more hetero atoms chosen from an oxygen, sulphur or nitrogen atom, and which may be substituted with one or more hydroxyl, C₁-C₆ alkoxy or amino radicals, and which may bear one or more ketone functions;
the ring members E, G, J, L and M, which may be identical or different, represent a carbon, oxygen, sulphur or nitrogen atom and form a ring chosen from pyridine, pyrimidine, pyrazine, triazine and pyridazine rings;
p is an integer between 0 and 3 inclusive;
m is an integer between 0 and 5 inclusive;
p+m is an integer between 0 and 5;
the radicals R₁₁, which may be identical or different, represent a halogen atom, a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a carboxyl radical, a C₁-C₆ alkylcarbonyl radical, a thio radical, a C₁-C₆ thioalkyl radical, a (C₁-C₆)alkylthio radical, an amino radical, an amino radical substituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical; it being understood that the radicals R₁₁ are borne by a carbon atom,
the radicals R₁₂, which may be identical or different, represent a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical, a C₁-C₆ carbamylalkyl radical, a (C₁-C₆)alkylcarboxy(C₁-C₆)alkyl radical or a benzyl radical; it being understood that the radicals R₁₂ are borne by a nitrogen atom,
R₁₃ represents a C₁-C₆ alkyl radical; a C₁-C₆ monohydroxyalkyl radical; a C₂-C₆ polyhydroxyalkyl radical; an aryl radical; a benzyl radical; a C₁-C₆ aminoalkyl radical; a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carboxyalkyl radical; a C₁-C₆ carbamylalkyl radical; a C₁-C₆ trifluoroalkyl radical; a tri(C₁-C₆)alkylsilane(C₁-C₆)alkyl radical; a C₁-C₆ sulphonamidoalkyl radical; a (C₁-C₆)alkylcarboxy(C₁-C₆)alkyl radical; a (C₁-C₆)alkylsulphinyl(C₁-C₆)alkyl radical; a (C₁-C₆)alkylsulphonyl(C₁-C₆)alkyl radical; a (C₁-C₆)alkylcarbonyl(C₁-C₆)alkyl radical; an N—(C₁-C₆)alkylcarbamyl(C₁-C₆)alkyl radical; an N—(C₁-C₆)alkylsulphonamido(C₁-C₆)alkyl radical;
x is 0 or 1
when x=0, the linker arm D is attached to the nitrogen atom,
when x=1, the linker arm D is attached to one of the ring members E, G, J, L or M,
Y is a counterion.

16. The composition of claim 15, wherein the ring members E, G, J, L and M form with the ring nitrogen a ring chosen from pyridine and pyrimidine rings.

17. The composition of claim 15, wherein the cationic tertiary para-phenylenediamine is such that x is equal to 0 and R₁₁ is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical mono- or disubstituted with a (C₁-C₆)alkyl, (C₁-C₆)alkylcarbonyl, amido or (C₁-C₆)alkylsulphonyl radical; a C₁-C₆ monohydroxyalkyl radical or a C₂-C₆ polyhydroxyalkyl radical, and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical or a C₁-C₆ carbamylalkyl radical.

18. The composition of claim 15, wherein the cationic tertiary para-phenylenediamine is such that x is equal to 1 and R₁₃ is chosen from a C₁-C₆ alkyl radical; a C₁-C₆ monohydroxyalkyl radical; a C₂-C₆ polyhydroxyalkyl radical; a C₁-C₆ aminoalkyl radical, a C₁-C₆ aminoalkyl radical in which the amine is mono- or disubstituted with a (C₁-C₆)alkyl, (C₁-C₆)alkylcarbonyl, amido or (C₁-C₆)alkylsulphonyl radical; a C₁-C₆ carbamylalkyl radical; a tri(C₁-C₆)alkylsilane(C₁-C₆)alkyl radical; a (C₁-C₆)alkylcarbonyl(C₁-C₆)alkyl radical; an N—(C₁-C₆)alkylcarbamyl(C₁-C₆)alkyl radical; R₁₁ is chosen from a hydroxyl radical, a C₁-C₆ alkyl radical, a C₁-C₆ monohydroxyalkyl radical, a C₂-C₆ polyhydroxyalkyl radical, a C₁-C₆ alkoxy radical, a tri(C₁-C₆)alkylsilane(C₁-C₆)alkyl radical, an amido radical, a C₁-C₆ alkylcarbonyl radical, an amino radical, an amino radical mono- or disubstituted with a (C₁-C₆)alkyl, (C₁-C₆)alkylcarbonyl, amido or (C₁-C₆)alkylsulphonyl radical; and R₁₂ is chosen from a C₁-C₆ alkyl radical, a C₁-C₆ monohydroxyalkyl radical, a C₂-C₆ polyhydroxyalkyl radical, a tri(C₁-C₆)alkylsilane(C₁-C₆)alkyl radical, a (C₁-C₆)alkoxy(C₁-C₆)alkyl radical and a C₁-C₆ carbamylalkyl radical.

19. The composition of claim 15, wherein the cationic tertiary para-phenylenediamine is such that R₁₁, R₁₂ and R₁₃ are alkyl radicals that may be substituted.

20. The composition of claim 2, wherein the cationic tertiary para-phenylenediamine is such that the radical R₂ is a radical of formula —XP(O)(O—)OCH₂CH₂N⁺(CH₃)₃ in which X represents an oxygen atom or a radical —NR₁₄, R₁₄ representing a hydrogen, a C₁-C₄ alkyl radical or a hydroxyalkyl radical.

21. The composition of claim 2, wherein the cationic tertiary para-phenylenediamine is such that the radical R₂ is a guanidine radical of formula —X—C═NR₈—NR₉R₁₀, X represents an oxygen atom or a radical —NR₁₁, R₈, R₉, R₁₀ and R₁₁ representing a hydrogen, a C₁-C₄ alkyl radical or a hydroxyalkyl radical.

22. The composition of claim 1, wherein the cationic tertiary para-phenylenediamine is chosen from the group formed by:
[1-(4-Aminophenyl)pyrrolidin-3-yl]trimethylammonium chloride;
[1-(4-Aminophenyl)pyrrolidin-3-yl]dimethyltetradecylammonium bromide;
N′-[1-(4-Aminophenyl)pyrrolidin-3-yl]-N,N-dimethylguanidinium chloride;
N-[1-(4-Aminophenyl)pyrrolidin-3-yl]guanidinium chloride;
3-[1-(4-Aminophenyl)pyrrolidin-3-yl]-1-methyl-3H-imidazol-1-ium chloride;
[1-(4-Aminophenyl)pyrrolidin-3-yl](2-hydroxyethyl)dimethylammonium chloride;
[1-(4-Aminophenyl)pyrrolidin-3-yl]dimethyl(3-trimethylsilanylpropyl)ammonium chloride;
[1-(4-Aminophenyl)pyrrolidin-3-yl](trrimethylammoniumhexyl)dimethylammonium dichloride
[1-(4-Aminophenyl)pyrrolidin-3-yl]oxophosphorylcholine
{2-[1-(4-Aminophenyl)pyrrolidin-3-yloxy]ethyl}-trimethylammonium chloride
1-{2-[1-(4-Aminophenyl)pyrrolidin-3-yloxy]ethyl}-1-methylpyrrolidinium chloride
3-{3-[1-(4-Aminophenyl)pyrrolidin-3-yloxy]propyl}-1-methyl-3H-imidazol-1-ium chloride
1-{2-[1-(4-Aminophenyl)pyrrolidin-3-yloxy]ethyl}-1-methylpiperidinium chloride
3-{3-[1-(5-Trimethylsilanylethyl-4-amino-3-trimethylsilanylethylphenyl)pyrrolidin-3-yloxy]propyl}-1-methyl-3H-imidazol-1-ium chloride
[1-(4-Amino-3-methylphenyl)pyrrolidin-3-yl]trimethylammonium chloride;
[1-(4-Amino-3-methylphenyl)pyrrolidin-3-yl]dimethyltetradecylammonium chloride;
N′-[1-(4-Amino-3-methylphenyl)pyrrolidin-3-yl]-N,N-dimethyl guanidinium chloride;
N-[1-(4-Amino-3-methylphenyl)pyrrolidin-3-yl]guanidinium chloride;
3-[1-(4-Amino-3-methylphenyl)pyrrolidin-3-yl]-1-methyl-3H-imidazol-1-ium chloride;
[1-(4-Amino-3-methylphenyl)pyrrolidin-3-yl](2-hydroxyethyl)dimethylammonium chloride;
[1-(4-Amino-3-methylphenyl)pyrrolidin-3-yl]dimethyl(3-trimethylsilanylpropyl ammonium chloride;
[1-(4-Amino-3-methylphenyl)pyrrolidin-3-yl](trrimethylammoniumhexyl)dimethylammonium dichloride
[1-(4-Amino-3-methylphenyl)pyrrolidin-3-yl]oxophosphorylcholine
{2-[1-(4-Amino-3-methylphenyl)pyrrolidin-3-yloxy]ethyl}-trimethylammonium chloride
1-{2-[1-(4-Amino-3-methylphenyl)pyrrolidin-3-yloxy]ethyl}-1-methylpyrrolidinium chloride
3-{3-[1-(4-Amino-3-methylphenyl)pyrrolidin-3-yloxy]propyl}-1-methyl-3H-imidazol-1-ium chloride
1-{2-[1-(4-Amino-3-methylphenyl)pyrrolidin-3-yloxy]ethyl}-1-methylpiperidinium chloride
[1-(4-Amino-3-trimethylsilanylethylphenyl)pyrrolidin-3-yl]trimethylammonium chloride
3-[1-(4-Amino-3-trimethylsilanylethylphenyl)pyrrolidin-3-yl]-1-methyl-3H-imidazol-1-ium chloride
3-{3-[1-(4-Amino-3-trimethylsilanylethylphenyl)pyrrolidin-3-yloxy]propyl}-1-methyl-3H-imidazol-1-ium chloride
[1-(5-Trimethylsilanylethyl-4-amino-3-trimethylsilanylethylphenyl)pyrrolidin-3-yl]trimethylammonium chloride
3-[1-(5-trimethylsilanylethyl-4-Amino-3-trimethylsilanylethylphenyl)pyrrolidin-3-yl]-1-methyl-3H-imidazol-1-ium chloride
1′-(4-Aminophenyl)-1-methyl[1,3′]bipyrrolidinyl-1-ium chloride;
1′-(4-Amino-3-methylphenyl)-1-methyl[1,3′]bipyrrolidinyl-1-ium chloride;
3-{[1-(4-Aminophenyl)pyrrolidin-3-ylcarbamoyl]methyl}-1-methyl-3H-imidazol-1-ium chloride;
3-{[1-(4-Aminophenyl)pyrrolidin-3-ylcarbamoyl]methyl}-1-methyl-3H-imidazol-1-ium chloride;
3-[1-(4-Aminophenyl)pyrrolidin-3-yl]-1-(3-trimethylsilanylpropyl)-3H-imidazol-1-ium chloride;
3-[1-(4-Amino-3-methylphenyl)pyrrolidin-3-yl]-1-(3-trimethylsilanylpropyl)-3H-imidazol-1-ium chloride;
[1-(4-Aminophenyl)pyrrolidin-3-yl]ethyldimethylammonium chloride;
[1-(4-Aminophenyl)pyrrolidin-3-yl]ethyldimethylammonium iodide;
[1-(4-Aminophenyl)pyrrolidin-3-yl]propyldimethylammonium iodide;
[1-(4-Aminophenyl)pyrrolidin-3-yl]propyldimethylammonium bromide;
[1-(4-Aminophenyl)pyrrolidin-3-yl]propyldimethylammonium methosulphate;
[1-(4-Aminophenyl)pyrrolidin-3-yl]butyldimethylammonium iodide;
[1-(4-Aminophenyl)pyrrolidin-3-yl]pentyldimethylammonium iodide;
[1-(4-Aminophenyl)pyrrolidin-3-yl]hexyldimethylammonium iodide;
[1-(4-Aminophenyl)pyrrolidin-3-yl]heptyldimethylammonium iodide;
[1-(4-Aminophenyl)pyrrolidin-3-yl]octyldimethylammonium iodide;
[1-(4-Aminophenyl)pyrrolidin-3-yl]decyldimethylammonium iodide;
[1-(4-Aminophenyl)pyrrolidin-3-yl]hexadecyldimethylammonium iodide;
[1-(4-Aminophenyl)pyrrolidin-3-yl]hydroxyethyldimethylammonium chloride;
[1-(4-Aminophenyl)pyrrolidin-3-yl]hydroxyethyldimethylammonium iodide.

23. The composition of claim 1, wherein the cationic tertiary para-phenylenediamine is chosen from the group formed by:
[1-(4-Aminophenyl)pyrrolidin-3-yl]trimethylammonium chloride;
[1-(4-Aminophenyl)pyrrolidin-3-yl]dimethyltetradecylammonium bromide;
N′-[1-(4-Aminophenyl)pyrrolidin-3-yl]-N,N-dimethyl guanidinium chloride;
N-[1-(4-Aminophenyl)pyrrolidin-3-yl]guanidinium chloride;
3-[1-(4-Aminophenyl)pyrrolidin-3-yl]-1-methyl-3H-imidazol-1-ium chloride;
[1-(4-Aminophenyl)pyrrolidin-3-yl](2-hydroxyethyl)dimethylammonium chloride;
[1-(4-Aminophenyl)pyrrolidin-3-yl]dimethyl(3-trimethylsilanylpropyl)ammonium chloride;
[1-(4-Amino-3-methylphenyl)pyrrolidin-3-yl]trimethylammonium chloride;
[1-(4-Amino-3-methylphenyl)pyrrolidin-3-yl]dimethyltetradecylammonium chloride;
N′-[1-(4-Amino-3-methylphenyl)pyrrolidin-3-yl]-N,N-dimethyl guanidinium chloride;
N-[1-(4-Amino-3-methylphenyl)pyrrolidin-3-yl]guanidinium chloride;
3-[1-(4-Amino-3-methylphenyl)pyrrolidin-3-yl]-1-methyl-3H-imidazol-1-ium chloride;
[1-(4-Amino-3-methylphenyl)pyrrolidin-3-yl](2-hydroxyethyl)dimethylammoniumchloride;
[1-(4-Amino-3-methylphenyl)pyrrolidin-3-yl]dimethyl(3-trimethylsilanylpropylammonium chloride;
1′-(4-Aminophenyl)-1-methyl[1,3′]bipyrrolidinyl-1-ium chloride;
1′-(4-Amino-3-methylphenyl)-1-methyl[1,3′]bipyrrolidinyl-1-ium chloride;
3-{[1-(4-Aminophenyl)pyrrolidin-3-ylcarbamoyl]methyl}-1-methyl-3H-imidazol-1-ium chloride;
3-{[1-(4-Amino-3-methylphenyl)pyrrolidin-3-ylcarbamoyl]methyl}-1-methyl-3H-imidazol-1-ium chloride;
3-[1-(4-Aminophenyl)pyrrolidin-3-yl]-1-(3-trimethylsilanylpropyl)-3H-imidazol-1-ium chloride;
3-[1-(4-Amino-3-methylphenyl)pyrrolidin-3-yl]-1-(3-trimethylsilanylpropyl)-3H-imidazol-1-ium chloride;
[1-(4-Aminophenyl)pyrrolidin-3-yl]ethyldimethylammonium chloride;
[1-(4-Aminophenyl)pyrrolidin-3-yl]ethyldimethylammonium iodide;
[1-(4-Aminophenyl)pyrrolidin-3-yl]propyldimethylammonium iodide;
[1-(4-Aminophenyl)pyrrolidin-3-yl]propyldimethylammonium bromide;
[1-(4-Aminophenyl)pyrrolidin-3-yl]propyldimethylammonium methosulphate;
[1-(4-Aminophenyl)pyrrolidin-3-yl]butyldimethylammonium iodide;
[1-(4-Aminophenyl)pyrrolidin-3-yl]pentyldimethylammonium iodide;
[1-(4-Aminophenyl)pyrrolidin-3-yl]hexyldimethylammonium iodide;
[1-(4-Aminophenyl)pyrrolidin-3-yl]heptyldimethylammonium iodide;
[1-(4-Aminophenyl)pyrrolidin-3-yl]octyldimethylammonium iodide;
[1-(4-Aminophenyl)pyrrolidin-3-yl]decyldimethylammonium iodide;
[1-(4-Aminophenyl)pyrrolidin-3-yl]hexadecyldimethylammonium iodide;
[1-(4-Aminophenyl)pyrrolidin-3-yl]hydroxyethyldimethylammonium chloride;
[1-(4-Aminophenyl)pyrrolidin-3-yl]hydroxyethyldimethylammonium iodide.

24. The composition of claim 1, wherein the cationic tertiary para-phenylenediamine is chosen from the group formed by:
[1-(4-Aminophenyl)pyrrolidin-3-yl]trimethylammonium chloride;
[1-(4-Aminophenyl)pyrrolidin-3-yl]dimethyltetradecylammonium bromide;
N′-[1-(4-Aminophenyl)pyrrolidin-3-yl]-N,N-dimethyl guanidinium chloride;
N-[1-(4-Aminophenyl)pyrrolidin-3-yl]guanidinium chloride;
3-[1-(4-Aminophenyl)pyrrolidin-3-yl]-1-methyl-3H-imidazol-1-ium chloride;
[1-(4-Aminophenyl)pyrrolidin-3-yl](2-hydroxyethyl)dimethylammonium chloride;
[1-(4-Aminophenyl)pyrrolidin-3-yl]dimethyl(3-trimethylsilanylpropyl)ammonium chloride;
[1-(4-Aminophenyl)pyrrolidin-3-yl](trimethylammoniumhexyl)dimethylammonium dichloride;
1′-(4-Aminophenyl)-1-methyl[1,3′]bipyrrolidinyl-1-ium chloride
3-[1-(4-Aminophenyl)pyrrolidin-3-yl]-1-(3-trimethylsilanylpropyl)-3H-imidazol-1-ium chloride;
3-[1-(4-Amino-3-methylphenyl)pyrrolidin-3-yl]-1-(3-trimethylsilanylpropyl)-3H-imidazol-1-ium chloride;
[1-(4-aminophenyl)pyrrolidin-3-yl]ethyldimethylammonium chloride;
[1-(4-Aminophenyl)pyrrolidin-3-yl]ethyldimethylammonium iodide;
[1-(4-Aminophenyl)pyrrolidin-3-yl]propyldimethylammonium iodide;
[1-(4-Aminophenyl)pyrrolidin-3-yl]propyldimethylammonium bromide;
[1-(4-Aminophenyl)pyrrolidin-3-yl]propyldimethylammonium methosulphate;
[1-(4-Aminophenyl)pyrrolidin-3-yl]butyldimethylammonium iodide;
[1-(4-Aminophenyl)pyrrolidin-3-yl]pentyldimethylammonium iodide;
[1-(4-Aminophenyl)pyrrolidin-3-yl]hexyldimethylammonium iodide;
[1-(4-Aminophenyl)pyrrolidin-3-yl]heptyldimethylammonium iodide;
[1-(4-Aminophenyl)pyrrolidin-3-yl]octyldimethylammonium iodide;
[1-(4-Aminophenyl)pyrrolidin-3-yl]decyldimethylammonium iodide;
[1-(4-Aminophenyl)pyrrolidin-3-yl]hexadecyldimethylammonium iodide;
[1-(4-Aminophenyl)pyrrolidin-3-yl]hydroxyethyldimethylammonium chloride;
[1-(4-Aminophenyl)pyrrolidin-3-yl]hydroxyethyldimethylammonium iodide.

25. The composition of claim 1, wherein the cationic tertiary para-phenylenediamine is chosen from the group formed by:
[1-(4-Aminophenyl)pyrrolidin-3-yl]trimethylammonium chloride;
3-[1-(4-Aminophenyl)pyrrolidin-3-yl]-1-methyl-3H-imidazol-1-ium chloride;
[1-(4-Aminophenyl)pyrrolidin-3-yl](2-hydroxyethyl)dimethylammonium chloride;
1′-(4-Aminophenyl)-1-methyl[1,3′]bipyrrolidinyl-1-ium chloride.

26. The composition of claim 1, wherein the cationic tertiary para-phenylenediamine is chosen from the group formed by:
[1-(4-Aminophenyl)pyrrolidin-3-yl]trimethylammonium chloride and [1-(4-Aminophenyl)pyrrolidin-3-yl](2-hydroxyethyl)dimethylammonium chloride.

27. The composition of claim 1, wherein the polyol ester is an ester of a saturated or unsaturated, linear or branched C₂-C₃₀ acid, whose hydrocarbon-based chain is optionally substituted with one or more hydroxyl groups, and of an optionally ethoxylated, linear, branched or cyclic C₂-C₅₀ polyol.

28. The composition of claim 27, wherein the polyol ester is an ester of an acid chosen from lauric acid, stearic acid, isostearic acid, oleic acid, ricinoleic acid.

29. The composition of claim 27, wherein the polyol ester is such that the polyol is chosen from ethylene glycol, propylene glycol, glycerol, glucose, sorbitol and sorbitol anhydride.

30. The composition of claim 27, wherein the polyol ester is chosen from ethylene glycol monostearate or distearate, glyceryl oleate or stearate, polyethylene glycol laurate, polyethylene glycol oleate and polyethylene glycol stearate.

31. The composition of claim 27, wherein the polyol ester is an ethoxylated polyol ester chosen from polyoxyethylenated ethers of glyceryl laureates, oleates and stearates and polyoxyethylenated ethers of sorbitol laurates and/or of sorbitol anhydride.

32. The composition of claim 31, wherein the number of ethylene oxides in the polyol esters is between 1 and 200.

33. The composition of claim 1, wherein the polyol ester(s) is (are) present in concentrations ranging from 0.01% to 20% and preferably from 0.05% to 10%.

34. The composition of claim 33, wherein the polyol ester(s) is (are) present in a concentration ranging from 0.1% to 5% by weight relative to the total weight of the composition.

35. The composition of claim 1, wherein the cationic tertiary para-phenylenediamine(s) containing a pyrrolidine nucleus represent(s) from 0.01% to 10% and preferably from 0.05% to 6% by weight relative to the total weight of the composition.

36. The composition of claim 1, wherein the composition further comprises at least one cationic polymer.

37. The composition of claim 1, wherein the composition further comprises at least one thickening polymer.

38. The composition of claim 1, wherein the composition further comprises at least one additional surfactant chosen from the group formed by anionic surfactants, amphoteric or zwitterionic surfactants, nonionic surfactants other than polyol esters and cationic surfactants.

39. The composition of claim 1, wherein the composition further comprises at least one additional oxidation base other than cationic tertiary para-phenylenediamines containing a pyrrolidine nucleus, chosen from para-phenylenediamines, bis(phenyl)alkylenediamines, para-aminophenols, ortho-aminophenols and heterocyclic bases, and the addition salts thereof.

40. The composition of claim 39, wherein the additional oxidation base(s) is (are) present in an amount of between 0.001% and 20% by weight and preferably between 0.005% and 6% by weight relative to the total weight of the composition.

41. The composition of claim 1, wherein the composition further comprises at least one coupler chosen from meta-phenylenediamines, meta-aminophenols, meta-diphenols, naphthalene-based couplers and heterocyclic couplers, and the addition salts thereof.

42. The composition of claim 41, wherein the coupler is chosen from 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

43. The composition of claim 41, wherein the coupler(s) is (are) present in an amount of between 0.001% and 20% and preferably between 0.005% and 6% by weight relative to the total weight of the composition.

44. The composition of claim 1, wherein the composition further comprises at least one direct dye.

45. The composition of claim 1, wherein the composition further comprises at least one hydroxylated solvent such as ethanol, propylene glycol, glycerol and polyol monoethers.

46. The composition of claim 1, wherein the composition further comprises an oxidizing agent chosen from hydrogen peroxide, urea peroxide, alkali metal bromates, persalts, peracids and oxydase enzymes, and preferably hydrogen peroxide.

47. A process for the oxidation dyeing of keratin fibres, wherein a dye composition as defined in claim 1 is applied to the fibres in the presence of an oxidizing agent.

48. A multi-compartment device, wherein a first compartment contains a dye composition for dyeing keratin fibres, as defined in claim 1, and a second compartment contains an oxidizing agent.
